(19) United States
(12) Patent Application Publication
(10) Pub. No.: US 2024/0117388 A1
(43) Pub. Date: Apr. 11, 2024

(54) ACYL ACTIVATING ENZYMES FOR PREPARATION OF CANNABINOIDS

(71) Applicant: BAYMEDICA, INC., Incline Village, NV (US)

(72) Inventors: James T. KEALEY, Incline Village, NV (US); James P. CRAIG, Incline Village, NV (US); Jianping SUN, Incline Village, NV (US); Charles K. MARLOWE, Incline Village, NV (US); Colin W. JOHNSON, Incline Village, NV (US); Philip J. BARR, Incline Village, NV (US)

(21) Appl. No.: 18/273,261

(22) PCT Filed: Jan. 20, 2022
(86) PCT No.: PCT/US22/13140

§ 371 Date: Jul. 19, 2023

Related U.S. Application Data
(60) Provisional application No. 63/139,689, filed on Jan. 20, 2021.

(51) Int. Cl. C12P 7/22; C12N 9/00; C12N 9/10; C12N 9/88; C12N 15/52; C12P 7/42

(52) U.S. Cl. CPC C12P 7/22; C12N 9/1085; C12N 9/13; C12N 9/88; C12N 9/93; C12N 15/52; C12P 7/42; C12Y 205/01102; C12Y 208/03001; C12Y 208/03008; C12Y 404/01026; C12Y 602/01012

(57) ABSTRACT

Enzymes and recombinant host cells for the biosynthesis of clinically important prenylated polyketides of the cannabinoid family are provided. Using readily available starting materials, heterologous enzymes (e.g., bacterial CoA-transferases and CoA-ligases) are used to direct cannabinoid biosynthesis in host cells such as recombinant yeast cells.

### CROSS-REFERENCES TO RELATED APPLICATIONS

The present application claims priority to U.S. Provisional Pat. Appl. No. 63/139,689, filed on Jan. 20, 2021, which application is incorporated herein by reference in its entirety.

### BACKGROUND OF THE INVENTION

Cannabis sativa varieties have been cultivated and utilized extensively throughout the world for a number of applications. Stems, branches, and leaves are used in fibers and fiber-based products; sprouts and seeds as food; seeds for inexpensive oils; flowers for aromatic, recreational, ritual and medicinal purposes; and flowers and roots for nutritional and additional medicinal and pharmaceutical applications. Indeed, many controlled clinical studies and anecdotal or open-label studies in humans have been documented that demonstrate beneficial effects of both plant extracts and purified C. sativa plant compounds in many human medical conditions. Beneficial activities of the cannabinoid family of compounds described from human studies range from neurological to mood/behavior disorders, and to gastrointestinal disorders as well as sleeping, appetite and fatigue problems. Other uses or potential uses include the treatment of various microbial and viral infections and the treatment of a number of cancers. Thus, as a direct result of this burgeoning list of human therapeutic indications, there currently exists an unfulfilled need for the production of pharmaceutical grade cannabinoids using sustainable, modern biopharmaceutical preparation methods.

Currently, the cannabinoids are isolated primarily via the cultivation of large acreages of cannabis or hemp plants in agricultural operations throughout the world, with a lower, albeit clinically important level of production methodologies that involve synthetic chemical processes. The former techniques are costly, utilize large quantities of natural resources, such as arable land and water and invariably lead to final pharmaceutical products that contain additional active cannabinoids that contaminate the desired active drug substances. This can lead to an inconsistency in the activities of the desired pure compounds leading to spurious activities in both clinical trial situations and in marketed products. Furthermore, the contamination of natural plant-derived cannabinoid preparations by toxic metals and pesticides is a problem that currently is in need of a solution. Also, because of the complex stereochemistry of many of the cannabinoids, chemical synthesis is a difficult, expensive and low-yielding process. Furthermore, the synthetic chemical production of a number of cannabinoids has been reported to produce less pharmacologically active molecules than those extracted from the C. sativa plant.

### BRIEF SUMMARY OF THE INVENTION

Provided herein are modified recombinant host cells engineered to produce cannabinoid products. The cells include a first exogenous polynucleotide that encodes a CoA-transferase or CoA-ligase that converts an aliphatic carboxylic acid to an acyl CoA thioester. Host cells may also contain further exogenous polynucleotides that encode a polyketide synthase (PKS), a 2-alkyl-4,6-dihydroxybenzoic acid cyclase, a prenyltransferase, and/or a cannabinoid synthase.

Also provided herein are methods of producing a cannabinoid product. The methods include:
culturing a modified recombinant host cell comprising a first exogenous polynucleotide that encodes a CoA-transferase or CoA-ligase that converts an aliphatic carboxylic acid to an acyl CoA thioester under conditions in which the CoA-transferase encoded by the exogenous polynucleotide is expressed and the acyl CoA thioester is produced; and
converting the acyl CoA thioester to the cannabinoid product.

In some embodiments, the recombinant host cells includes:
a second exogenous polynucleotide that encodes a polyketide synthase (PKS) that produces a polyketide from the acyl CoA thioester and malonyl CoA,
a third exogenous polynucleotide that encodes a 2-alkyl-4,6-dihydroxybenzoic acid cyclase, and/or
a fourth polynucleotide that encodes a prenyltransferase;
wherein culturing the modified recombinant host cell comprises expressing products encoded by the second, third, and/or fourth exogenous polynucleotides,
converting the acyl CoA thioester to a 2-alkyl-4,6-dihydroxybenzoic acid or a 5-alkyl-benzene-1,3-diol, and/or
converting the 2-alkyl-4,6-dihydroxybenzoic acid or the 5-alkyl-benzene-1,3-diol to a prenylated 2-alkyl-4,6-dihydroxybenzoic acid or a prenylated 5-alkyl-benzene-1,3-diol.

### BRIEF DESCRIPTION OF THE DRAWINGS

FIG. 1 shows a biosynthetic scheme for preparation of CBGA and CBGVA.

### DETAILED DESCRIPTION OF THE INVENTION

The present invention is based, in part on the discovery that exogenous CoA-transferases (e.g., from bacteria such as Roseburia hominis) and CoA-ligases can be employed in recombinant yeast cells or other host cells for production of a variety naturally-occurring cannabinoids and non-natural cannabinoid analogs.

### I. DEFINITIONS

Unless otherwise defined, all terms of art, notations and other scientific terminology used herein are intended to have the meanings commonly understood by those of ordinary skill in the art to which the present application pertains. In some cases, terms with commonly understood meanings are defined herein for clarity and/or for ready reference, and the inclusion of such definitions herein should not necessarily be construed to represent a substantial difference over what is generally understood in the art.

As used herein, the terms “cannabinoid,” “cannabinoid compound,” and “cannabinoid product” are used interchangeably to refer to a molecule containing a polyketide moiety, e.g., olivetolic acid or another 2-alkyl-4,6-dihydroxybenzoic acid, and a terpene-derived moiety e.g., a geranyl group. Geranyl groups are derived from the diphosphate of geraniol, known as geranyl pyrophosphate, which can react with olivetolic acid type compounds to form the acidic cannabinoid cannabigerolic acid (CBGA) and CBGA analogs. CBGA can be converted to further bioactive cannabinoids both enzymatically (e.g., by decarboxylation via enzyme treatment in vivo or in vitro) and chemically (e.g., by heating).

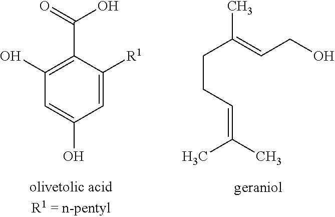

The term cannabinoid includes acid cannabinoids and neutral cannabinoids. The term “acidic cannabinoid” refers to a cannabinoid having a carboxylic acid moiety. The carboxylic acid moiety may be present in protonated form (i.e., as —COOH) or in deprotonated form (i.e., as carboxylate —COO-). Examples of acidic cannabinoids include, but are not limited to, cannabigerolic acid, cannabidiolic acid, cannabichromenic acid and Δ9-tetrahydrocannabinolic acid. The term “neutral cannabinoid” refers to a cannabinoid that does not contain a carboxylic acid moiety (i.e., does not contain a moiety —COOH or —COO-). Examples of neutral cannabinoids include, but are not limited to, cannabigerol, cannabidiol, cannabichromene and Δ9-tetrahydrocannabinol.

The term “2-alkyl-4,6-dihydroxybenzoic acid” refers to a compound having the structure:

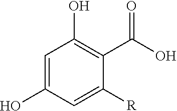

wherein R is a C₁-C₂₀ alkyl group, which in some embodiments, can be halogenated, hydroxylated, deuterated, and/or tritiated. Examples of 2-alkyl-4,6-dihydroxybenzoic acids include, but are not limited to olivetolic acid (i.e., 2-pentyl-4,6-dihydroxybenzoic acid; CAS Registry No. 491-72-5) and divarinic acid (i.e., 2-propyl-4,6-dihydroxybenzoic acid; CAS Registry No. 4707-50-0). Olivetolic acid analogs include other 2-alkyl-4,6-dihydroxybenzoic acids and substituted resorcinols including, but not limited to, 5-halomethylresorcinols, 5-haloethylresorcinols, 5-halopropylresorcinols, 5-halohexylresorcinols, 5-haloheptylresorcinols, 5-halooctylresorcinols, and 5-halononylresorcinols.

The term “prenyl moiety” refers to a substituent containing at least one methylbutenyl group (e.g., a 2-methylbut-2-ene-1-yl group). In many instances prenyl moieties are synthesized biochemically from isopentenyl pyrophosphate and/or isopentenyl diphosphate giving rise to terpene natural products and other compounds. Examples of prenyl moieties include, but are not limited to, prenyl, geranyl, myrcenyl, ocimenyl, farnesyl, and geranylgeranyl.

The term “geraniol” refers to (2E)-3,7-dimethyl-2,6-octadien-1-ol (CAS Registry No. 106-24-1). The term “geranylating” refers to the covalent bonding of a 3,7-dimethyl-2,6-octadien-1-yl radical to a molecule such as a 2-alkyl-4,6-hydroxybenzoic acid. Geranylation can be conducted chemically or enzymatically, as described herein.

The term “2-alkyl-4,6-dihydroxybenzoic acid” refers to a compound having the structure:

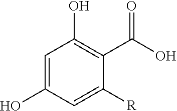

wherein R is a C₁-C₂₀ alkyl group. Examples of 2-alkyl-4,6-dihydroxybenzoic acids include, but are not limited to olivetolic acid (i.e., 2-pentyl-4,6-dihydroxybenzoic acid; CAS Registry No. 491-72-5) and divarinic acid (i.e., 2-propyl-4,6-dihydroxybenzoic acid; CAS Registry No. 4707-50-0). Olivetolic acid analogs include other 2-alkyl-4,6-dihydroxybenzoic acids and substituted resorcinols such as 5-methylresorcinol, 5-ethylresorcinol, 5-propylresorcinol, 5-hexylresorcinol, 5-heptylresorcinol, 5-octylresorcinol, and 5-nonylresorcinol.

The term “alkyl,” by itself or as part of another substituent, refers to a straight or branched, saturated, aliphatic radical. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc.

The term “alkenyl,” by itself or as part of another substituent, refers to an alkyl group, as defined herein, having one or more carbon-carbon double bonds. Examples of alkenyl groups include, but are not limited to, vinyl (i.e., ethenyl), crotyl (i.e., but-2-en-1-yl), penta-1,3-dien-1-yl, and the like. Alkenyl moieties may be further substituted, e.g., with aryl substituents (such as phenyl or hydroxyphenyl, in the case of 4-hydroxystyryl).

The terms “halogen” and “halo,” by themselves or as part of another substituent, refer to a fluorine, chlorine, bromine, or iodine atom.

The term “haloalkyl,” by itself or as part of another substituent, refers to an alkyl group where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

The term “hydroxyalkyl,” by itself or as part of another substituent, refers to an alkyl group where some or all of the hydrogen atoms are replaced with hydroxyl groups (i.e., —OH groups). As for alkyl and haloalkyl groups, hydroxyalkyl groups can have any suitable number of carbon atoms, such as C₁₋₆.

The term “deuterated” refers to a substituent (e.g., an alkyl group) having one or more deuterium atoms (i.e., ²H atoms) in place of one or more hydrogen atoms.

The term “tritiated” refers to a substituent (e.g., an alkyl group) having one or more tritium atoms (i.e., ³H atoms) in place of one or more hydrogen atoms.

An “organic solvent” refers to a carbon-containing substance that is liquid at ambient temperature and pressure and is substantially free of water. Examples of organic solvents include, but are not limited to, toluene, methylene chloride, ethyl acetate, acetonitrile, tetrahydrofuran, benzene, chloroform, diethyl ether, dimethyl formamide, dimethyl sulfoxide, and petroleum ether.

The term “acid” refers to a substance that is capable of donating a proton (i.e., a hydrogen cation) to form a conjugate base of the acid. Examples of acids include, but are not limited to, mineral acids (e.g., hydrochloric acid, sulfuric acid, and the like), carboxylic acids (e.g., acetic acid, formic acid, and the like), and sulfonic acids (e.g., methanesulfonic acid, p-toluenesulfonic acid, and the like).

Throughout this specification and claims, the word “comprise,” or variations such as “comprises” or “comprising,” will be understood to imply the inclusion of a stated integer or group of integers but not the exclusion of any other integer or group of integers.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percent amino acid sequence identity can be performed in various methods, including those using publicly available computer software such as BLAST, BLAST-2, ALIGN, Geneious, or Megalign (DNASTAR) software, among others. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, BLAST 2.0 can be used with the default parameters described to determine percent sequence identity.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) aliphatic amino acids Gly, Ala, Val, Leu and Ile; (vi) slightly polar amino acids Met and Cys; (vii) small-side chain amino acids Ser, Thr, Asp, Asn, Gly, Ala, Glu, Gln and Pro; (viii) small hydroxyl amino acids Ser and Thr; and sulfur-containing amino acids Cys and Met. Reference to the charge of an amino acid in this paragraph refers to the charge at pH 7.0.

In specific cases, abbreviated terms are used. For example, the term “CBGA” refers to cannabigerolic acid. Likewise: “OA” refers to olivetolic acid; “CBG” refers to cannabigerol; “CBDA” refers to cannabidiolic acid; “CBD” refers to cannabidiol; “THC” refers to Δ⁹-tetrahydrocannabinol (Δ⁹-THC); “Δ⁸-THC” refers to Δ⁸-tetrahydrocannabinol; “THCA” refers to Δ⁹-tetrahydrocannabinolic acid (Δ⁹-THCA); “Δ⁸-THCA” refers to Δ⁸-tetrahydrocannabinolic acid; “CBCA” refers to cannabichromenic acid; “CBC” refers to cannabichromene; “CBN” refers to cannabinol; “CBND” refers to cannabinodiol; “CBNA” refers to cannabinolic acid; “CBV” refers to cannabivarin; “CBVA” refers to cannabivarinic acid; “THCV” refers to Δ⁹-tetrahydrocannabivarin (Δ⁹-THCV); “Δ⁸-THCV” refers to “Δ⁸-tetrahydrocannabivarin; “THCVA” refers to Δ⁹-tetrahydrocannabivarinic acid (Δ⁹-THCV); “Δ⁸-THCVA” refers to Δ⁸-tetrahydrocannabivarinic acid; “CBGV” refers to cannabigerovarin; “CBGVA” refers to cannabigerovarinic acid; “CBCV” refers to cannabichromevarin; “CBCVA” refers to cannabichromevarinic acid; “CBDV” refers to cannabidivarin; “CBDVA” refers to cannabidivarinic acid; “MPF” refers to multiple precursor feeding; “PKS” refers to a polyketide synthase; “GOT” refers to geranyl pyrophosphate olivetolate geranyl transferase; “YAC” refers to yeast artificial chromosome; “IRES” or “internal ribosome entry site” means a specialized sequence that directly promotes ribosome binding and mRNA translation, independent of a cap structure; and “HPLC” refers to high performance liquid chromatography.

As used herein and in the appended claims, the singular forms “a,” “and,” and “the” include plural referents unless the context clearly dictates otherwise.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

Certain methodologies employed here are described, for example, in Green et al., Molecular Cloning: A Laboratory Manual 4th. edition (2012) Cold Spring Harbor Laboratory Press, Cold Spring Harbor, N. Y.; and Ausubel, et al., Current Protocols in Molecular Biology, through Jul. 17, 2018, John Wiley & Sons, Inc. As appropriate, procedures involving the use of commercially available kits and reagents are generally carried out in accordance with manufacturer defined protocols and/or parameters unless otherwise noted. Before the present methods, expression systems, and uses therefore are described, it is to be understood that this invention is not limited to the particular methodology, protocols, cell lines, organism species or genera, constructs, and reagents described as such may, of course, vary. It is also to be understood that the terminology used herein is for the purpose of describing particular embodiments only, and is not intended to limit the scope of the present invention, which will be limited only by the appended claims.

### II. CANNABINOID EXPRESSION SYSTEMS

Cannabinoid compounds of interest and cannabinoid compound intermediates are produced using an expression system as described herein that employs a CoA-transferase or a CoA-ligase. Such compounds include, without limitation, CBG, CBDA, CBD, THC, Δ⁸-THC, THCA, Δ⁸-THCA, CBCA, CBA, CBN, CBDN, CBNA, CBV, CBVA, THCV, THCVA, Δ⁸-THCA, CBGV, CBGVA, CBCV, CBCVA, CBDV and CBDVA; as well as compounds including, but not limited to, the cannabichromanones, cannabicoumaronone, cannabicitran, 10-oxo-Δ^{6a(10a)}-tetrahydrohydrocannabinol (OTHC), cannabiglendol, and Δ⁷-isotetrahydrocannabinol, as well as analogs of such compounds, e.g., halogenated or deuterated compounds. In some embodiments, each step of a metabolic pathway that produces the cannabinoid compound of interests occurs in a modified recombinant cell described herein. In other embodiments, at least one step of the metabolic pathway occurs in a modified recombinant cell described herein, and at least one step of the metabolic pathway occurs extracellularly, e.g., in yeast media or within a co-cultured modified recombinant cell. The compounds produced at each step of the metabolic pathway may be referred to as “intermediates” or “intermediate compounds” or “compound intermediates.”

In some embodiments, host cells genetically modified to express an exogenous CoA-transferase or an exogenous CoA-ligase are provided. In some embodiments, the CoA-transferase is an acetate CoA-transferase or a propionate CoA-transferase In some embodiments, the host cells are additionally modified to express an exogenous polyketide synthase, an exogenous 2-alkyl-4,6-dihydroxybenzoic acid cyclase, an exogenous prenyltransferase, and/or an exogenous cannabinoid synthase.

### A. CoA-Transferases

Examples of organisms that express CoA-transferases for use in the methods can be found in the Comprehensive Enzyme Information System (BRENDA) under Enzyme Commission numbers EC 2.8.3.8 (acetate CoA-transferase) and EC 2.8.3.1 (propionate CoA-transferase). These organisms include, but are not limited to: Anaerostipes species and strains (e.g., A. caccae; A. caccae DSM 14662), Anaerobutyricum species and strains (e.g., A. hallii; A. hallii M72/1), Anaerotignum species and strains (e.g., A. propionicum), Aspergillus species and strains (e.g., A. nidulans), Butyrivibrio species and strains (e.g., B. fibrisolvens; B. fibrisolvens 16/4), Clostridium species and strains (e.g., C. kluyveri), Coprococcus species and strains (e.g., Coprococcus sp. L2-50), Cupriavidus species (e.g., C. necator H16), Escherichia species and strains (e.g., E. coli K-12); Eubacterium species and strains (e.g., E. rectale, E. rectale DSM 17629), Faecalibacterium species and strains (e.g., F. prausnitzii; F. prausnitzii A2-165; F. prausnitzii L2-6; F. prausnitzii M21/2), Megasphaera species and strains (e.g., M. elsdenii), Propionibacterium strains and species (e.g., P. freudenreichii), and Roseburia species and strains (e.g., R. hominis, R. intestinalis; R. intestinalis L1-82; R. inulinivorans; R. inulinivorans A2-194; and Roseburia sp. A2-181). Non-limiting examples of specific CoA-transferases are listed in Table 1.

TABLE 1
CoA-Transferases for use in preparation of cannabinoids.

| | | UniProt |
|---|---|---|
| Organism | CoA-transferase | Accession No. |
| A. caccae DSM 14662 | Butyryl-CoA:acetate CoA-transferase | B0MC58 |
| A. hallii | Butyryl-CoA:acetate CoA-transferase | D2WEY8 |
| A. propionicum | Propionate CoA-transferase | Q9L3F7 |
| A. nidulans | Propionate CoA-transferase | |
| B. fibrisolvens | Butyryl-CoA:acetate CoA-transferase | D2WEY7 |
| C. kluyveri | Propionate CoA-transferase | |
| C. necator | Propionate CoA-transferase | |
| C. necator H16 | Acetate CoA-transferase YdiF | Q0K874 |
| E. coli K-12 | Acetyl-CoA:acetoacetate CoA- | P76459 |
| | transferase subunit beta (AtoA) | |
| E. coli K-12 | Acetyl-CoA:acetoacetate-CoA- | P76458 |
| | transferase subunit alpha (AtoD) | |
| E. rectale DSM 17629 | Butyryl-CoA:acetate CoA-transferase | D2WEY1 |
| F. prausnitzii | Butyryl-CoA:acetate CoA-transferase | D2WEZ2 |
| F. prausnitzii M21/2 | Butyryl-CoA:acetate CoA-transferase | A8SFP6 |
| F. prausnitzii A2-165 | Butyryl-CoA:acetate CoA-transferase | C7H5K4 |
| M. elsdenii | Propionate CoA-transferase | |
| P. freudenreichii | Propionate CoA-transferase | |
| R. hominis DSM 16839 | Butyryl-CoA:acetate CoA-transferase | G2SYC0 |
| R. intestinalis L1-82 | Butyryl-CoA:acetate CoA-transferase | C7GB37 |
| R. inulinivorans | Butyryl-CoA:acetate CoA-transferase | D2WEY6 |

In some embodiments, the CoA-transferase is selected from the group consisting of R. hominis butyryl-CoA:acetate CoA-transferase, E. coli acetyl-CoA:acetoacetyl-CoA-transferase, and C. necator H16 propionate CoA-transferase.

In some embodiments, the CoA-transferase comprises an R. hominis butyryl-CoA:acetate CoA-transferase polypeptide sequence, e.g., as set forth in SEQ ID NO:1. In some embodiments, the CoA-transferase comprises E. coli acetyl-CoA:acetoacetyl-CoA transferase polypeptide sequences, e.g., as set forth in SEQ ID NO:2 and SEQ ID NO:3. In some embodiments, the CoA-transferase comprises a C. necator H16 propionate CoA-transferase polypeptide sequence, e.g., as set forth in SEQ ID NO:4. In some embodiments, the CoA-transferase comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:1 or 4. In some embodiments, the CoA-transferase comprises amino acid sequences that have at least 60% or greater identity to the sequences set forth in SEQ ID NOS:2 and 3. In some embodiments, the CoA-transferase has at least 70%, 75%, 80%, 85%, 90%, 95%, or greater identity to the sequence set forth in SEQ ID NO:1 or 4. In some embodiments, the CoA-transferase comprises polypeptides having at least 70%, 75%, 80%, 85%, 90%, 95%, or greater identity to the sequences set forth in SEQ ID NOS:2 and 3. In some embodiments, the CoA-transferase comprises the amino acid sequence of SEQ ID NO:1 or 4. In some embodiments, the CoA-transferase comprises the amino acid sequences of SEQ ID NOS: 2 and 3.

### B. CoA-Ligases

In some embodiments, the CoA-ligase is selected from the group consisting of M. avium mig medium chain acyl-CoA-ligase and A. thaliana AT4g05160 coumarate acyl-CoA-ligase.

In some embodiments, the CoA-ligase is selected from the group consisting of M. avium mig medium chain acyl-CoA-ligase, A. thaliana AT4g05160 coumarate acyl-CoA-ligase, S. cerevisiae FAA2 medium chain acyl-CoA-ligase, and E. coli FADK acyl-CoA-ligase.

In some embodiments, the CoA-ligase comprises an M. avium mig medium chain acyl-CoA-ligase polypeptide sequence, e.g., as set forth in SEQ ID NO:5. In some embodiments, the CoA-ligase comprises an A. thaliana AT4g05160 coumarate acyl-CoA-ligase polypeptide sequence, e.g., as set forth in SEQ ID NO:6. In some embodiments, the CoA-ligase comprises a S. cerevisiae FAA2 medium chain acyl-CoA-ligase polypeptide sequence, e.g., as set forth in SEQ ID NO:7. In some embodiments, the CoA-ligase comprises an E. coli FADK acyl-CoA-ligase polypeptide sequence, e.g., as set forth in SEQ ID NO:8. In some embodiments, the CoA-ligase comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:5, 6, 7, or 8. In some embodiments, the CoA-ligase has at least 70%, 75%, 80%, 85%, 90%, 95%, or greater identity to the sequence set forth in SEQ ID NO:5, 6, 7, or 8. In some embodiments, the CoA-ligase comprises the amino acid sequence of SEQ ID NO:5, 6, 7, or 8.

### C. Polyketide Synthases

In the some embodiments, the PKS is a type I PKS, a type II PKS, or a type III PKS. Examples of PKSs include, but are not limited to, those described in WO 2018/209143 and WO 2020/102430, which are incorporated herein by reference in their entirety.

### Type I PKS

In some embodiments, a host cell is genetically modified to express an exogenous polynucleotide that encodes a Type I PKS or a non-naturally occurring variant of a Type I PKS that has polyketide synthase activity. In some embodiments, the Type I PKS is an iterative partially reducing PKS. Partially reducing PKSs share a highly conserved domain architecture that distinguishes them from non-reducing and highly reducing PKSs in that although they may have a ketoreductase (KR) domain, they lack dehydratase or enoyl-reductase domains for further reductive processing. In some embodiments, Type I PKS polypeptides are selected to employ hexanoyl-CoA as a starter unit.

Type I PKSs that can be preferentially utilized include PKSs that are naturally initiated by a starter unit hexanoyl-CoA such as the PKS encoding the micacocidin biosynthetic pathway or, alternatively, iterative Type I PKSs such as orsellinic acid synthase (OSAS), or 6-methylsalicylic acid synthase (6-MSAS) that have been mutated to accept longer chain fatty acid starter units to produce olivetolic and divarinic acids and their analogs.

In exemplary embodiments, the exogenous Type I PKS is an iterative partially reducing PKS that produces the antibiotic micacocidin and is derived from the bacterium Ralstonia solanacearum (Kage et al., Chemistry and Biology 20:764-771, 2013; Kage et al., Org. Biomol. Chem. 13:11414-11417, 2015).

The MicC PKS of Ralstonia solanacearum comprises a loading module followed by three extender modules. In some embodiments of a genetically modified host cell as described herein, the Type I PKS encoded by an exogenous polynucleotide comprises the loading module and extender module 1 of MicC, which comprises the following domains: an adenylation (A₁) domain, an acyl carrier protein (ACP) domain, a ketosynthase (KS) domain, an acyl transferase (AT) domain, a KR domain, and an ACP domain at the C-terminal end of 5 the module. In some embodiments, the PKS comprises a MicC polypeptide sequence, e.g., as set forth in SEQ ID NO:9. In some embodiments, the KR domain is inactivated by mutation at the active site of the KR domain, e.g., by mutation of the Tyr at position 1991, which is part of a catalytic triad together with Lys and Ser residues (see, e.g., Caffrey, ChemBioChem 4:654-657, 2003). In some embodiments, a phenylalanine is introduced to substitute for the Tyr at position 1991. In other embodiments, an aliphatic amino acid residue, e.g., alanine, is substituted for Tyr at position 1991.

In some embodiments, the exogenous polynucleotide encodes a Type I PKS that comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:9. In some embodiments, the polynucleotide encodes a Type I PKS polypeptide that has at least 70%, 75%, 80%, 85%, 90%, 95%, or greater, identity to the sequence set forth in SEQ ID NO:9. In some embodiments, the Type I PKS comprises a polypeptide sequence that is a non-naturally occurring variant of SEQ ID NO:9. In some embodiments, the variant comprises a mutation in the KR domain that inactivates the KR domain. In some embodiments, the PKS comprises a polypeptide sequence as set forth in SEQ ID NO:9 in which the tyrosine at position 1991, as determined with reference to SEQ ID NO:9, comprises a substitution, e.g., an alanine substitution that inactivates the KR domain.

In some embodiments, the genetically modified host cell is further engineered to express a phosphopantetheinyl transferase (PPTase). In particular embodiments, the PPTase gene is MicA from Ralstonia solanacearum, or an ortholog thereof, e.g., from another Ralstonia species. In some embodiments, the PPTase comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:10. In some embodiments, the polynucleotide encodes a PPTase that has at least 70%, 75%, 80%, 85%, 90%, 95%, or greater, identity to the sequence set forth in SEQ ID NO:10. In some embodiments, the PPTase comprises the amino acid sequence of SEQ ID NO:10. In alternative embodiments, the PPTase is a fungal or bacterial PPTase, e.g., NpgA or sfp.

In some embodiments the Type I PKS is a mutant orsellinic acid synthase derived from Aspergillus nidulans (orsA) or from Fusarium graminearum (PKS14). For example, the SAT domain of the OSAS Orsa or of PKS14 can be replaced with the SAT domain of PksA or BenQ.

### Type II PKS

In some embodiments, a host cell is genetically modified to express an exogenous polynucleotide that encodes a Type II PKS or a non-naturally occurring variant of a Type II PKS that has polyketide synthase activity. In some embodiments, the Type II PKS encodes a PKS that can use hexanoyl CoA as a starter unit. In some embodiments, the Type II PKS comprises a BenA polypeptide or a multimeric BenA-BenB-BenC PKS enzyme from a Streptomyces sp., or an ortholog thereof, that naturally produces benastatin. As used herein, a “BenA PKS” refers to a PKS comprising BenA encoded by the BenA gene of the benastatin gene cluster. In some embodiments, a “BenA PKS” additionally contains BenB and BenC.

In some embodiment the exogenous polynucleotide encodes a Type II PKS that comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:11. In some embodiments, the polynucleotide encodes a Type II PKS polypeptide that has at least 70%, 75%, 80%, 85%, 90%, 95%, or greater, identity to the sequence set forth in SEQ ID NO:11. In some embodiments, the Type II PKS comprises a polypeptide sequence that is a non-naturally occurring variant of SEQ ID NO:11.

In some embodiments, the genetically modified host cell is further engineered to express BenQ, a FabH-like ketoacyl-synthase (KASIII), which plays a role in providing and selecting hexanoate as the PKS starter unit. In particular embodiments, the polynucleotide introduced in the genetically modified host cell comprises a nucleic acid sequence that encodes BenQ from a Streptomyces sp., or an ortholog thereof. In some embodiments, the BenQ polypeptide comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:12. In some embodiments, the polynucleotide encodes a BenQ polypeptide that has at least 70%, 75%, 80%, 85%, 90%, 95%, or greater, identity to the sequence set forth in SEQ ID NO:12. In some embodiments, the BenQ polypeptide comprises the amino acid sequence of SEQ ID NO:12.

In some embodiments, the host cell is genetically modified to express a multimeric BenA-BenB-BenC PKS enzyme. In some embodiments, the polynucleotide introduced in the genetically modified host cell comprises a nucleic acid sequence that encodes BenB from a Streptomyces sp, or an ortholog thereof. In some embodiments, the BenB polypeptide comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:13. In some embodiments, the polynucleotide encodes a BenB polypeptide that has at least 70%, 75%, 80%, 85%, 90%, 95%, or greater, identity to the sequence set forth in SEQ ID NO:13. In some embodiments, the BenB polypeptide comprises the amino acid sequence of SEQ ID NO:13. In further embodiments, the polynucleotide introduced in the genetically modified host cell comprises a nucleic acid sequence that encodes BenC from a Streptomyces sp, or an ortholog thereof. In some embodiments, the BenC polypeptide comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:14. In some embodiments, the polynucleotide encodes a BenC polypeptide that has at least 70%, 75%, 80%, 85%, 90%, 95%, or greater, identity to the sequence set forth in SEQ ID NO:14. In some embodiments, the BenC polypeptide comprises the amino acid sequence of SEQ ID NO:14.

### Type III PKS

In some embodiments, the PKSs employed are from the type III class of PKS, e.g., the natural aromatic olivetolic acid synthase/cyclase systems, or the related type III orsellinic acid synthases, or modified versions of these enzymes. Olivetolic acid synthase (Taura et al. FEBS Letters 583:2061-2066, 2009), also referred to as 3, 5, 7,-trioxododecanoyl-CoA synthase, UniProtKB-B1Q2B6, is a type III PKS that that catalyzes the condensation of acyl-CoAs with three molecules of malonyl-CoA to form a 3,5,7-trioxoalkanoyl-CoA tetraketide as shown below:

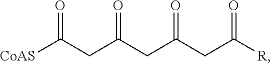

wherein “CoA” is coenzyme A and “R” is an alkyl group. For example, when hexanoic acid is used as the starting feed for cannabinoid production, the hexanoyl-CoA formed by the CoA-transferase or CoA-ligase as described above is condensed with three molecules of malonyl-CoA to form 3,5,7-trioxododecanoyl-CoA (i.e., “R” is an n-pentyl group). Type III PKSs are homodimeric enzymes that act directly on acyl-CoA substrates (as opposed to acyl carrier protein-bound substrates, in the case of type I PKSs and type II PKSs). Type III PKSs are well characterized, for example, by Yu et al. (IUBMB Life, 64(4): 285-295, 2012).

In some embodiments, the type III PKS comprises a olivetolic acid synthase polypeptide sequence having about 60% or greater identity (e.g., about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:15. In some embodiments, the type III PKS comprises an olivetolic acid synthase polypeptide sequence having about 70%, 75%, 80%, 85%, 90%, 95%, or greater identity to the sequence set forth in SEQ ID NO:15.

### D. 2-Alkyl-4,6-Dihydroxybenzoic Acid Cyclases

Host cells may be further modified to express an exogenous polynucleotide that encodes a 2-alkyl-4,6-dihydroxybenzoic acid cyclase (e.g., olivetolic acid cyclase). In some embodiments, the 2-alkyl-4,6-dihydroxybenzoic acid cyclase is a dimeric α+β barrel (DABB) protein domain that resembles DABB-type polyketide cyclases from Streptomyces. Olivetolic acid cyclase is described, for example, by Gagne et al. (Proc. Nat. Acad. Sci. USA 109 (31): 12811-12816; 2012). The term “2-alkyl-4,6-dihydroxybenzoic acid cyclase” includes variants, e.g., a truncated or modified polypeptide, that have cyclase activity; and naturally occurring homologs or orthologs. In some embodiments, the 2-alkyl-4,6-dihydroxybenzoic acid cyclase is olivetolic acid cyclase from C. sativa (EC number 4.4.1.26). In some embodiments, the 2-alkyl-4,6-dihydroxybenzoic acid cyclase produces divarinic acid (see, e.g., Yang et al., FEBS J. 283:1088-1106, 2016). In some embodiments, the 2-alkyl-4,6-dihydroxybenzoic acid cyclase is an olivetolic acid cyclase homolog from Arabidopsis thaliana AtHS1 (Uniprot Q9LUV2, see also Yang et al., supra), Populus tremula SP1 (P0A881), A. thaliana At5g22580 (Q9FK81), S. glaucescens TcmI cyclase (P39890), S. coelicolor ActVA-Orf6 (Q53908), P. reinekei MLMI (C5MR76), S. nogalater SnoaB (054259), M. tuberculosis Rv0793 (086332), or P. aeruginosa PA3566 (Q9HY51). In some embodiments, the cyclase is the N-terminal domain of a BenH protein from a benastatin gene cluster, e.g., from Streptomyces sp. A2991200. In some embodiments, the 2-alkyl group of the 2-alkyl-4,6-dihydroxybenzoic acid contains 1-18 carbon atoms. In some embodiments, the 2-alkyl group of the 2-alkyl-4,6-dihydroxybenzoic acid contains 1-12 carbon atoms. In some embodiments, the 2-alkyl group of the 2-alkyl-4,6-dihydroxybenzoic acid contains 1-9 carbon atoms.

In the some embodiments, the 2-alkyl-4,6-dihydroxybenzoic acid cyclase is olivetolic acid cyclase, an AtHS1 polypeptide, or the N-terminal domain of a BenH polypeptide.

In some embodiments, the polynucleotide encoding the 2-alkyl-4,6-dihydroxybenzoic acid cyclase encodes a polypeptide that has 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:16, 17, or 18. In some embodiments, the polypeptide has at least 70%, 75%, 80%, 85%, 90%, 95%, or greater identity to the sequence set forth in SEQ ID NO:16, 17, or 18.

In some embodiments, the polynucleotide encoding the 2-alkyl-4,6-dihydroxybenzoic acid cyclase encodes an a polypeptide has 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:19. In some embodiments, the polypeptide has at least 70%, 75%, 80%, 85%, 90%, 95%, or greater identity to the sequence set forth in SEQ ID NO:19.

In some embodiments, the polynucleotide encoding the 2-alkyl-4,6-dihydroxybenzoic acid cyclase encodes an a polypeptide has 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:20. In some embodiments, the polypeptide has at least 70%, 75%, 80%, 85%, 90%, 95%, or greater identity to the sequence set forth in SEQ ID NO:20.

### E. Prenyltransferases

In the some embodiments, the modified recombinant host cell further comprises fourth polynucleotide that encodes a prenyltransferase for production of prenylated 2-alkyl-4,6-dihydroxybenzoic acids or prenylated 5-alkyl-benzene-1,3-diols.

Examples of prenyltransferases include, but are not limited to, those described in WO 2018/209143 and U.S. Provisional Pat. Appl. No. 62/963,448, which are incorporated herein by reference in their entirety. In some embodiments, the prenyltransferase may be geranylpyrophosphate:olivetolate geranyltransferase (GOT; EC 2.5.1.102) as described by Fellermeier & Zenk (FEBS Letters 427:283-285; 1998). Streptomyces prenyltransferases including NphB, as described by Kumano et al. (Bioorg Med Chem. 16(17): 8117-8126; 2008), can also be employed. In some embodiments, the prenyltransferase is fnq26, i.e., flaviolin linalyltransferase from Streptomyces cinnamonensis. In some embodiments, a host cell genetically modified to express the prenyltransferase may be a modified host cell as described in the following below. In some embodiments, the yeast host cells are modified to express a GOT for catalyzing the coupling of geranyl-pyrophosphate to olivetolic acid. In some embodiments, the amino acid sequence of the GOT is SEQ ID NO:21. In some embodiments, the GOT polypeptide comprises an amino acid sequence of SEQ ID NO:22.

In some embodiments, multiple copies of a polynucleotide encoding a prenyltransferase are integrated into the host cell genome. For example, 5-20 copies (e.g., 5-15 copies or 5-10 copies) may be integrated at various positions into the genome of a yeast cell. Various loci, including those which encode non-essential genes, are suitable for integration of prenyltransferase-encoding polynucleotides. One or more copies may be integrated at each loci. In some embodiments, two copies of the polynucleotide are directionally arranged at one, two, or three loci and one copy of the polynucleotide is integrated at one two or three other loci. In some embodiments, two or more copies of the prenyltransferase-encoding polynucleotide are integrated at one or more of the S. cerevisiae loci YEL060C, YHR090C, YER024W, YIL114C, and YHR162W. In some embodiments, 5-10 (e.g., 9) copies of the prenyltransferase-encoding polynucleotide are integrated at the S. cerevisiae loci YEL060C, YHR090C, YER024W, YIL114C, and YHR162W.

Exogenous prenyl species, such as geraniol, can be supplied to the host cells during culture and production of the prenylated compounds. Alternatively, the host cells can be cultured in media containing high levels of prenyl precursors, e.g., prenol, isoprenol, geraniol, and the like. In procedures including multiple precursor feeding (MPF), 5-carbon prenol and isoprenol can be enzymatically converted to the monophosphate level (i.e., to dimethylallyl monophosphate and isopentenyl monophosphate) and then to the diphosphate level (i.e., to dimethylallyl pyrophosphate and isopentenyl pyrophosphate) prior to coupling to form the 10-carbon geranyl pyrophosphate.

Thus, as detailed herein, in some embodiments relating to the biosynthesis of an initiating aromatic polyketide precursor, enzymes that form simple starting units are expressed and used to generate, from exogenously supplied aliphatic carboxylic acids, acylthioesters, typically acetyl-, propionyl-, butanoyl-, hexanoyl-, malonyl- or methylmalonyl-coenzyme-A (CoA) thioesters. These are then condensed repeatedly with malonyl-CoA to form the aromatic polyketide building blocks for the next step in cannabinoid biosynthesis, namely prenylation.

In some embodiments, modified recombinant host cells are also provided, which host cells comprise an exogenous polynucleotide that encodes prenol and isoprenol kinase; an exogenous polynucleotide that encodes kinase activity to produce dimethylallyl pyrophosphate and isopentenyl pyrophosphate when grown in the presence of exogenous prenol and isoprenol; an exogenous polynucleotide that encodes a geranyl-pyrophosphate synthase; and and/or an exogenous polynucleotide that encodes a prenyltransferase that catalyzes coupling of geranyl-pyrophosphate to olivetolic acid or an olivetolic acid analog (e.g., a 2-alkyl-4,6-dihydroxybenzoic acid) to form a cannabinoid compound. In some embodiments, the 2-alkyl group of the 2-alkyl-4,6-dihydroxybenzoic acid contains 1-18 carbon atoms. In some embodiments, the 2-alkyl group of the 2-alkyl-4,6-dihydroxybenzoic acid contains 1-12 carbon atoms. In some embodiments, the 2-alkyl group of the 2-alkyl-4,6-dihydroxybenzoic acid contains 1-9 carbon atoms.

Five-carbon prenols (prenol and isoprenol) may be converted by several enzymes to the monophosphate level and then to the diphosphate level by additional expressed enzymes, prior to their coupling to give the 10-carbon geranyl-diphosphate by the enzyme GPP-synthase. In some embodiments, the initial kinase event is performed by the enzyme hydroxyethylthiazole kinase. This enzyme has been described in several organisms from where the encoding genes are derived, including E. coli, Bacillus subtilis, Rhizobium leguminosarum, Pyrococcus horikoshii, S. cerevisiae and maize species.

Further phosphorylation to the diphosphate level is achieved by using the enzyme isoprenyl diphosphate synthase or isopentenylphosphate kinase, see U.S. Pat. No. 6,235,514. In some embodiments, chemically synthesized genes encoding this enzyme or more active mutants are derived by using the Thermoplasma acidophilum, Methanothermobacter thermautotrophicus, Methano-caldococcus jannaschii, Mentha x piperita or Mangifera indica amino acid sequences, or other homologous sequences with kinase activity.

The 10-carbon geranyl-diphosphate may also be generated by a kinase that phosphorylates geraniol to the monophosphate level, followed by a second kinase that gives rise to geranyl-diphosphate. In some embodiments, the first kinase event is performed by the enzyme farnesol kinase (FOLK) (Fitzpatrick, Bhandari and Crowell, 2011; Plant J. 2011 Jun;66(6):1078-88). This kinase enzyme is derived from the known amino acid sequences or mutants from the organisms that phosphorylate the 5-carbon prenols, including plants (Arabidopsis thaliana, Camelina sativa, Capsella rubella, Noccaea caerulescens etc.) and fungi (Candida albicans, Talaromyces atroroseus, etc.).

Further phosphorylation of geranyl-phosphate to the geranyl-diphosphate level is achieved by using a mutated enzyme isopentenyl monophosphate kinase (IPK) Mutations in IPK (Va173, Va1130, Ile140) have been reported to give rise to enhanced geranyl-phosphate kinase activity (Mabanglo et al., 2012). This kinase enzyme is derived from the known amino acid sequences or mutants from bacteria or archaeal species, including but not limited to Methanocaldococcus jannaschii, and Thermoplasma acidophilum.

In some embodiments, the DNA construct for the geranyl diphosphate:olivetolate geranyltransferase encodes the wild type or a mutant enzyme with yeast-preferred codons. In others, DNA constructs that encode bacterial, e.g., Streptomyces prenyltransferases with relaxed substrate specificities are used (Kumano et al., 2008).

In some embodiments, the host cell comprises one or more additional exogenous polynucleotides selected from the three following exogenous polynucleotides: an exogenous polynucleotide that encodes a prenol and isoprenol kinase; an exogenous polynucleotide that encodes a kinase that produces dimethylallyl pyrophosphate and isopentenyl pyrophosphate when grown in the presence of exogenous prenol and isoprenol; and an exogenous polynucleotide that encodes a geranyl-pyrophosphate synthase.

In some embodiments, the high aqueous solubility of both prenol and isoprenol is leveraged together with recombinant host cells that express heterologous kinase enzymes that can phosphorylate these 5-carbon compounds to the diphosphate level, thereby trapping them, due to the charged diphosphate moieties, within the host cell.

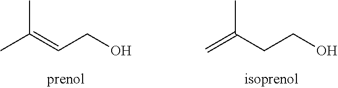

In some embodiments, the resulting diphosphates are then condensed to form geranyl-diphosphate (also referred to a geranyl-pyrophosphate) through the action of either endogenous or heterologously expressed geranyl-pyrophosphate synthase (GPP synthase). This is then available for condensation with a 2-alkyl-4,6-dihydroxybenzoic acid through the action of a wild type or preferably a more active mutant aromatic prenyltransferase enzyme to form cannabigerolic acid or a cannabigerolic acid analog.

In other embodiments, geraniol itself is converted, through the actions of heterologously expressed kinase enzymes to form geranyl-pyrophosphate, which is then coupled with olivetolic acid or an olivetolic acid analog (e.g., 2-alkyl-4,6-dihydroxybenzoic acid), through the action of a wild-type prenyltransferase or a mutant prenyltransferase enzyme, to form cannabigerolic acid or a cannabigerolic acid analog.

In some embodiments, host cells are further modified to express a CBDA synthase (EC 1.21.3.8), a THCA synthase, or CBCA synthase as further described below.

In the some embodiments, the prenyltransferase is geranylpyrophosphate:olivetolate geranyltransferase.

In the some embodiments, expression of one or more of the exogenous polynucleotides is driven by an ADH2 promoter, an ADH1 promoter, a GAL1 promoter, a MET25 promoter, a CUP1 promoter, a GPD promoter, a PGK promoter, a PYK promoter, a TPI promoter, a TEF1 promoter, or an FBA1 promoter.

In the some embodiments, at least two of the exogenous polynucleotides are present in the same autonomously replicating expression vector and expressed as a multicistronic mRNA.

### F. Cannabinoid Synthases

In some embodiments, recombinant host cells are further modified to convert a cannabinoid product such as cannabigerolic acid to further cannabinoids. In some such embodiments, the expression system is on the same vector or on a separate vector, or is integrated into the host cell genome. In other embodiments, the expression system for the conversion activity encodes one of the C. sativa enzymes CBCA synthase, THCA synthase, or CBDA synthase. In some embodiments, the synthase is a homolog from hops, e.g., a CBDA synthase homolog from hops. In some embodiments, the expression system encode a hops CBDA homolog that has at least 70% identity or at least 75%, identity, or at least about 80% or greater identity (e.g., about 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:23, 24, or 25. In some embodiments, the polypeptide has about 75%, 80%, 85%, 90%, 95%, or greater identity to the sequence set forth in SEQ ID NO:23, 24, or 25.

CBCAS can be expressed as a fusion protein lacking its own signal peptide, or can be expressed with its own signal peptide or a heterologous signal peptide or hydrophobic domain at its amino terminus.

In other embodiments, an HDEL or KDEL endoplasmic reticulum-retention sequence is fused to the expressed GOT, CBCAS or GOT/CBCAS mutant enzymes. In some embodiments, the GOT and CBCAS constructs may be modified to introduce targeted mutations, or random mutations in the expressed enzymes, such that the expressed enzyme has favorable properties for cannabinoid acid production.

In some embodiments, the CBCAS signal peptide is the endogenous signal peptide used by the cannabis plant, or it may be replaced by a yeast or a heterologous targeting sequence such as the yeast alpha-factor pre- or pre-pro- sequence, the yeast proteinase A pre- or pre-pro- sequence, or sequences derived from the Cannabis GOT (which is also referred to here as an “CsPT4”) enzyme such as for example, the hydrophobic region(s) starting around amino acid 80 of the mature GOT3 enzyme. Other preferred signal peptides include the S. cerevisiae pdi1 signal sequence or the berberine bridge-associated easE signal sequence from Aspergillus japonica. The CBCAS gene construct may be modified by changing the sequence to remove N-linked glycosylation sites in the protein. All permutations and combinations of glycosylation site modifications may be examined for increased or optimal activities. In other embodiments, a fusion protein, such as hSOD may be incorporated into the constructs to be expressed. CBCA, THCA or CBDA synthase gene constructs may be similarly modified.

Illustrative CBCAS polypeptide sequences are provided in SEQ ID NOS:26-32. In some embodiments, the polynucleotide encoding the CBCAS encodes a polypeptide that has at least 70% identity, or at least 75% identity, or at least about 80% or greater identity (e.g., about 81%, 82%, 83%, 84%, 85%, 86%, 87%, 88%, 89%, 90%, 91%, 92%, 93%, 94%, 95%, 96%, 97%, 98%, 99%, or 100% identity) to the regions of the CBCAS polypeptide of any one of SEQ ID NOS:26-32 that excludes the signal sequence or ER-retention sequence. In some embodiments, the polypeptide has about 75%, 80%, 85%, 90%, 95%, or greater identity to the sequence set forth in any one of SEQ ID NOS:26-32.

### Engineering the Host Cell

Polynucleotides can be introduced into host cells using any methodology. In some embodiments, exogenous polynucleotides encoding two or more enzymes (e.g., two of: a CoA-transferase or CoA-ligase; a Type I polyketide synthase, Type II polyketide synthase, or Type III polyketide synthase; and a 2-alkyl-4,6-dihydroxybenzoic acid cyclase) as described herein are present in the same expression construct, e.g., an autonomously replicating expression vector. In some embodiments, two or more of the enzymes are expressed as components of a multicistronic RNA in which expression is driven by the same promoter. Thus, for example, in some embodiments, an exogenous polynucleotide encoding a CoA-transferase and an exogenous polynucleotide encoding a PKS, a 2-alkyl-4,6-dihydroxybenzoic acid cyclase, or a prenyltransferase may be contained in an expression construct driven by the same promoter. In some embodiments, an expression vector, e.g., an autonomously replicating vector, may comprise two exogenous polynucleotides for generating a cannabinoid separated by an internal ribosome entry site (IRES) such that expression is driven by the same promoter to generate a dicistronic mRNA. In some embodiments, the promoter is an alcohol dehydrogenase-2 promoter. In some embodiments, exogenous polynucleotides are present in the same expression construct, e.g., an autonomously replicating expression vector, and are operably linked to separate promoters. In some embodiments, exogenous polynucleotides are present in two or more expression constructs, e.g., autonomously replicating expression vectors. In some embodiments, the autonomously replicating expression vector is a yeast artificial chromosome. In some embodiments, one or more of the exogenous polynucleotides are integrated into the host genome. In some embodiments, multiple exogenous polynucleotides are introduced into the host cell by retrotransposon integration.

### Host Cells

In some embodiments, the host cell is a yeast or a filamentous fungus host cell such as an Aspergillus host cell. Genera of yeast that can be employed as host cells include, but are not limited to, cells of Saccharomyces, Schizosaccharomyces, Candida, Hansenula, Pichia, Kluyveromyces, Yarrowia and Phaffia. Suitable yeast species include, but are not limited to, Saccharomyces cerevisiae, Schizosaccharomyces pombe, Candida albicans, Hansenula polymorpha, Pichia pastoris, P. canadensis, Kluyveromyces marxianus, Kluyveromyces lactis, Phaffia rhodozyma and, Yarrowia lipolytica. Filamentous fungal genera that can be employed as host cells include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma. Illustrative species of filamentous fungal species include Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

In the some embodiments, the modified recombinant host cell is a cell selected from the group consisting of Saccharomyces cerevisiae, Kluyveromyces lactis, Kluyveromyces marxianus, Pichia pastoris, Yarrowia lipolytica, Hansenula polymorpha and Aspergillus.

In some embodiments, the yeast strain is a modified industrial ethanol producing strain and/or is strain “Super alcohol active dry yeast” (Angel Yeast Co., Ltd. Yichang, Hubei 443003, P.R. China). Such strains are modified by curing to cir⁰ and have selectable markers (e.g., URA3 and LEU2) integrated into the genome. Additional yeast strains that can be used include InvSc1 (MATa his3Δ1 leu2 trp1-289 ura3-52/MATa his3Δ1 leu2 trp1-289 ura3-5) (Invitrogen), or the protease deficient strain BJ2168 (ATCC 208277 MATa prc1-407 prb1-1122 pep4-3 leu2 trp1 ura3-52 ga12).

In the above embodiments, the genes may be encoded by chemically synthesized genes, with yeast codon optimization, that encode a wild type or mutant enzyme from C. sativa, R. hominis, E. coli, C. necator, M avium, A. thaliana or other organism.

Promoters used for driving transcription of genes in S. cerevisiae and other yeasts include, but are not limited to, DNA elements that are regulated by glucose concentration in the growth media, such as the alcohol dehydrogenase-2 (ADH2) promoter. Other regulated promoters or inducible promoters, such as those that drive expression of the GAL1, MET25 and CUP1 genes, are used when conditional expression is required. GAL1 and CUP1 are induced by galactose and copper, respectively, whereas MET25 is induced by the absence of methionine.

In some embodiments, one or more of the exogenous polynucleotides is operably linked to a glucose regulated promoter. In some embodiments, expression of one or more of the exogenous polynucleotides is driven by an alcohol dehydrogenase-2 promoter.

Other promoters drive strongly transcription in a constitutive manner. Such promoters include, without limitation, the control elements for highly expressed yeast glycolytic enzymes, such as glyceraldehyde-3-phosphate dehydrogenase (GPD), phosphoglycerate kinase (PGK), pyruvate kinase (PYK), triose phosphate isomerase (TPI), enolase (ENO2), and alcohol dehydrogenase-1 (ADH1). Other strong constitutive promoters that may be used are those from the S. cerevisiae transcription elongation factor EF-1 alpha genes (TEF1 and TEF2) (Partow et al., Yeast. 2010, (11):955-64; Peng et al., Microb Cell Fact. 2015, (14):91-102) and the high-affinity glucose transporter (HXT7) and chaperonin (SSA1) promoters that function well under conditions of low glucose following the S. cerevisiae diauxic shift (Peng et al., Microb Cell Fact. 2015, (14):91-102).

In some embodiments, the host cells can increase cannabinoid production by increasing precursor pools and the like. Heterologous natural or chemically synthesized genes for enzymes such as malonyl-CoA synthase, with malonate feeding (Mutka et al., FEMS Yeast Res. 2006), and acetyl-CoA carboxylases 1 and 2 up-regulate the important malonyl-CoA for PKS biosynthesis. Similarly, acetyl-CoA synthases -1 and -2, and other gene products in the mevalonate pathway, e.g., acetoacetyl-CoA thiolase or the NphT7 gene product from Streptomyces sp. (Okamura et al., Proc Natl Acad Sci USA. 2010), HMG-CoA synthase, mevalonate kinase, phosphomevalonate kinase, mevalonate diphosphate decarboxylase, isopentenyl diphosphate: dimethylallyl diphosphate isomerase, HMG-CoA reductase, mutant farnesyl-pyrophosphate synthase (ERG20; Zhao et al., 2016) from Saccharomyces or other eukaryotic species may also be introduced on high-level expression plasmid vectors or through genomic integration using methods well known to those skilled in the art. Such methods may involve CRISPR Cas-9 technology, yeast artificial chromosomes (YACs) or the use of retrotransposons. Alternatively, if natural to the host organism, such genes may be up-regulated by genetic element integration methods known to those skilled in the art.

In some embodiments, similar engineering may be employed to reduce the production of natural products, e.g., ethanol that utilize carbon sources that lead to reduced utilization of that carbon source for cannabinoid production. Such genes may be completely “knocked out” of the genome by deletion, or may be reduced in activity through reduction of promoter strength or the like. Such genes include those for the enzymes ADH1 and/or ADH6. Other gene “knockouts” include genes involved in the ergosterol pathway, such as ERGS and the two most prominent aromatic decarboxylase genes of yeast, PAD1 and FDC1.

In some embodiments, yeast strains that overexpress integrated genes or modified genes of the mevalonate pathway are utilized to biosynthesize geranyl-diphosphate for production of cannabinoids. In some embodiments, host cells are engineered to overexpress one or more enzymes of the mevalonic acid pathway. Such enzymes, includes, for example, Erg10, Erg13, HMGR, Erg 12, Erg8, Mvd1, Idi1, and Erg 20. See, e.g., U.S. Pat. No. 6,689,593, which is incorporated by reference. In some embodiments, yeast strains for cannabinoid production have has the following integrations using S. cerevisiae-based sequences unless otherwise noted:

At the HO locus: pTEF1-IDI1; pADH2-tHMGR; pADH2-ERG13; pTEF2-ERG20 (F96W, N127W); at the YFL041W locus: pMLS1-ERG20 (F96W, N127W); pICL1-ERG13; pADH2 (S. para)-tHMGR; pFBA1-MatB (S. co); at the REI1 locus: pMLS1-ERG12; pFB A1-MVD1; pADH2-mvaE (E. fa); pICL1-mvaS (E. fa); pTEF1-ERG8; at the PRB1 locus: pURA3-URA3; pTEF1-ADR1; pFBA1-PDC (Z. mo); at the YER180C locus: pTDH3-FAD synthetase (S. ce); pTEF1-Hac1 (Pichia CAY67758.1); pFBA1-calnexin (Pichia CAY68938.1); at the PEP4 locus: pADH2-Erg9; pSSA1-GPPS (Abies grandis).

In some embodiments, host cells are modified to include genes for accessory enzymes aimed at assisting in the production of the final product cannabinoids. One such enzyme, catalase, is able to neutralize hydrogen peroxide produced by certain enzymes involved in the oxido-cyclization of CBGA and analogs, such as cannabidiolic acid synthase (Taura et al., 2007), Δ⁹-tetrahydrocannabinolic acid synthase (Sirikantaramas et al., 2004) and cannabichromenic acid synthase (Morimoto et al., 1998).

In some embodiments, the engineered host cells contain up-regulated or down-regulated endogenous or heterologous genes to optimize, for example, the precursor pools for cannabinoid biosynthesis. Additional, further heterologous gene products may be expressed to give “accessory” functions within the cell. For example, overexpressed catalase may be expressed in order to neutralize hydrogen peroxide formed in the oxido-cyclization step to important acidic cannabinoids such as CBDA, Δ⁹-THCA and CBCA. “Accessory” genes and their expressed products may be provided through integration into the yeast genome through techniques well known in the art, or may be expressed from plasmids (also known as yeast expression vectors), yeast artificial chromosomes (YACs) or yeast transposons.

Recombinant host cells may be made by transforming a host cell, either through genomic integration or using episomal plasmids (also referred to as expression vectors, or simply vectors) with at least one nucleotide sequence encoding enzymes involved in the engineered metabolic pathways. The term “nucleotide sequence,” “nucleic acid sequence,” and “genetic construct” are used interchangeably to refer to a polymer of RNA or DNA, single- or double-stranded, optionally containing synthetic, non-natural or altered nucleotide bases. A nucleotide sequence may comprise one or more segments of cDNA, genomic DNA, synthetic DNA, or RNA. In some embodiments, the nucleotide sequence is codon-optimized to reflect the typical codon usage of the host cell without altering the polypeptide encoded by the nucleotide sequence. In certain embodiments, the term “codon optimization” or “codon-optimized” refers to modifying the codon content of a nucleic acid sequence without modifying the sequence of the polypeptide encoded by the nucleic acid to enhance expression in a particular host cell. In certain embodiments, the term is meant to encompass modifying the codon content of a nucleic acid sequence as a means to control the level of expression of a polypeptide (e.g., either increase or decrease the level of expression). Accordingly, described are nucleic sequences encoding the enzymes involved in the engineered metabolic pathways. In some embodiments, a metabolically engineered cell may express one or more polypeptide having an enzymatic activity necessary to perform the steps described below. In some embodiments, the nucleotide sequences are synthesized and codon-optimized for expression in yeast according to methods described in U.S. Pat. No. 7,561,972.

For example a particular cell may comprises one, two, three, four, five or more than five nucleic acid sequences, each one encoding the polypeptide(s) necessary to produce a cannabinoid compound, or cannabinoid compound intermediate described herein. Alternatively, a single nucleic acid molecule can encode one, or more than one, polypeptide. For example, a single nucleic acid molecule can contain nucleic acid sequences that encode two, three, four or even five different polypeptides. Nucleic acid sequences may be obtained from a variety of sources such as, for example, amplification of cDNA sequences, DNA libraries, de novo synthesis, excision of genomic segment. The sequences obtained from such sources may then be modified using standard molecular biology and/or recombinant DNA technology to produce nucleic sequences having desired modifications. Exemplary methods for modification of nucleic acid sequences include, for example, site directed mutagenesis, PCR mutagenesis, deletion, insertion, substitution, swapping portions of the sequence using restriction enzymes, optionally in combination with ligation, homologous recombination, site specific recombination or various combination thereof. In other embodiments, the nucleic acid sequences may be a synthetic nucleic acid sequence. Synthetic polynucleotide sequences may be produced using a variety of methods described in U.S. Pat. No. 7,323,320, as well as U.S. Pat. Appl. Pub. Nos. 2006/0160138 and 2007/0269870. Methods of transformation of yeast cells are well known in the art.

### III. METHODS FOR CANNABINOID PRODUCTION

Also provided herein are methods of producing a cannabinoid product. The methods include:
culturing a modified recombinant host cell comprising a first exogenous polynucleotide that encodes a CoA-transferase or CoA-ligase that converts an aliphatic carboxylic acid to an acyl CoA thioester under conditions in which the CoA-transferase encoded by the exogenous polynucleotide is expressed and the acyl CoA thioester is produced; and
converting the acyl CoA thioester to the cannabinoid product.

In some embodiments, the CoA-transferase is selected from the group consisting of R. hominis butyryl-CoA:acetate CoA-transferase, E. coli acetyl-CoA:acetoacetyl-CoA-transferase, and C. necator H16 propionate CoA-transferase. In some embodiments, the CoA-ligase is selected from the group consisting of M. avium mig medium chain acyl-CoA-ligase, and A. thaliana AT4g05160 coumarate acyl-CoA-ligase.

In the some embodiments, the aliphatic carboxylic acid is a C₂₋₅ carboxylic acid (e.g., acetic acid, propionic acid, butyric acid, etc.) or a C₆₋₂₀ carboxylic acid (e.g., hexanoic acid, heptanoic acid, octanoic acid, etc.). In the some embodiments, the aliphatic carboxylic acid comprises a carbon-carbon double bond, a hydroxy group, a halogen, deuterium, tritium, or a combination thereof. The aliphatic carboxylic acid may be, for example, a compound according to Formula I:

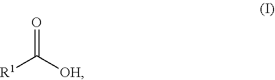

wherein R¹ is selected from the group consisting of hydrogen, C₁-C₂₀ alkyl, C₁-C₂₀ haloalkyl, C₁-C₂₀ hydroxyalkyl, deuterated C₁-C₂₀ alkyl, tritiated C₁-C₂₀ alkyl, and C₂-C₂₀ alkenyl. In some embodiments, R¹ is selected from the group consisting of C₁-C₁₀ haloalkyl, C₁-C₁₀ hydroxyalkyl, deuterated C₁-C₁₀ alkyl, tritiated C₁-C₁₀ alkyl, or C₂-C₁₀ alkenyl. In some embodiments, the carboxylic acid is selected from the group consisting of 4-fluorobutanoic acid, 5-fluoropentanoic acid, and 6-fluorohexanoic acid.

In the some embodiments, the modified recombinant host cell further comprises:
a second exogenous polynucleotide that encodes a polyketide synthase (PKS) that produces a polyketide from the acyl CoA thioester and malonyl CoA, and/or
a third exogenous polynucleotide that encodes a 2-alkyl-4,6-dihydroxybenzoic acid cyclase;
wherein culturing the modified recombinant host cell comprises expressing products encoded by the second and third exogenous polynucleotides and converting the acyl CoA thioester to a 2-alkyl-4,6-dihydroxybenzoic acid or a 5-alkyl-benzene-1,3-diol; and
wherein converting the acyl CoA thioester to the cannabinoid product comprises converting the 2-alkyl-4,6-dihydroxybenzoic acid or the 5-alkyl-benzene-1,3-diol to the cannabinoid product.

Any of the CoA-transferases, CoA-ligases, PKSs, and 2-alkyl-4,6-dihydroxybenzoic acid cyclases described above may be used in a number of suitable combinations. In some embodiments, the CoA-transferase is R. hominis butyryl-CoA:acetate CoA-transferase, the aliphatic carboxylic acid is butyric acid, and the 2-alkyl-4,6-dihydroxybenzoic acid is divarinic acid.

In some embodiments, the methods include production of a 2-alkyl-4,6-dihydroxybenzoic acid 5-or alkylbenzene-1,3-diol according to Formula II:

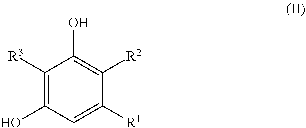

wherein:
R¹ is selected from the group consisting of C₁-C₂₀ alkyl, C₁-C₂₀ haloalkyl, C₁-C₂₀ hydroxyalkyl, deuterated C₁-C₂₀ alkyl, tritiated C₁-C₂₀ alkyl, and C₂-C₂₀ alkenyl,
R² is selected from the group consisting of COOR^{2a} and H,
R^{2a} is selected from the group consisting of H and C₁-C₆ alkyl, and
R³ is selected from the group consisting of a prenyl moiety and H.

### Fermentation Conditions

Cannabinoid production according to the methods provided herein generally includes the culturing of host cells (e.g., yeast or filamentous fungi) that have been engineered to contain the expression systems described above. In some embodiments, the carbon sources for yeast growth are sugars such as glucose, dextrose, xylose, or other sustainable feedstock sugars such as those derived from cellulosic sources, for example. In other embodiments, the carbon sources used may be methanol, glycerol, ethanol or acetate. In some embodiments, feedstock compositions are refined by experimentation to provide for optimal yeast growth and final cannabinoid production levels, as measured using analytical techniques such as HPLC. In such embodiments, methods include utilization of glucose/ethanol or glucose/acetate mixtures wherein the molar ratio of glucose to the 2-carbon source (ethanol or acetate) is between the ranges of 50/50, 60/40, 80/20, or 90/10. Feeding may be optimized to both induce glucose-regulated promoters and to maximize the production of acetyl-CoA and malonyl-CoA precursors in the production strain.

Fermentation methods may be adapted to a particular yeast strain due to differences in their carbon utilization pathway or mode of expression control. For example, a Saccharomyces yeast fermentation may require a single glucose feed, complex nitrogen source (e.g., casein hydrolysates), and multiple vitamin supplementation. This is in contrast to the methylotrophic yeast Pichia pastoris which may require glycerol, methanol, and trace mineral feeds, but only simple ammonium (nitrogen) salts, for optimal growth and expression. See, e.g., Elliott et al. J. Protein Chem. (1990) 9:95 104, U.S. Pat. No. 5,324,639 and Fieschko et al. Biotechnol. Bioeng. (1987) 29:1113 1121. Culture media may contain components such as yeast extract, peptone, and the like. The microorganisms can be cultured in conventional fermentation modes, which include, but are not limited to, batch, fed-batch, and continuous flow.

In some embodiments, the rate of glucose addition to the fermenter is controlled such that the rate of glucose addition is approximately equal to the rate of glucose consumption by the yeast; under such conditions, the amount of glucose or ethanol does not accumulate appreciably. The rate of glucose addition in such instances can depend on factors including, but not limited to, the particular yeast strain, the fermentation temperature, and the physical dimensions of the fermentation apparatus.

For the MPF procedure, in batch mode, the precursors olivetolic acid (or an olivetolic acid analog such as another 2-alkyl-4,6-dihydroxybenzoic acid), olivetol (or an olivetol analog such as another 5-alkylbenzene-1,3-diol), prenol, isoprenol or geraniol may be present in concentrations of between 0.1 and 50 grams/L (e.g., between 1 and 10 g/L). In fed-batch mode, the precursors may be fed slowly into the fermentation over between 2 and hours, such that a final addition of between 1 and 100 grams/L (e.g., between 1 and 10 grams/L, or between 10 and 100 grams/L) of each requisite precursor occurs.

Similarly, carboxylic acid starting materials such as hexanoic acid, butanoic acid, pentanoic acid, and the like may be present in concentrations of between 0.1 and 50 grams/L (e.g., between 1 and 10 g/L). In fed-batch mode, the carboxylic acid may be fed slowly into the fermentation over between 2 and 20 hours, such that a final addition of between 1 and 100 grams/L (e.g., between 1 and 10 grams/L, or between 10 and 100 grams/L) of the carboxylic acid occurs.

Culture conditions such as expression time, temperature, and pH can be controlled so as to afford target cannabinoid intermediates (e.g., olivetolic acid) and/or target cannabinoid products (e.g., CBGA, CBG) in high yield. Host cells are generally cultured in the presence of starting materials, such as hexanoic acid, prenol, isoprenol, or the like, for periods of time ranging from a few hours to a day or longer (e.g., 24 hours, 30 hours, 36 hours, or 48 hours) at temperatures ranging from about 20° C. to about 40° C. depending on the particular host cells employed. For example, S. cerevisiae may be cultured at 25-32° C. for 24-40 hours (e.g., 30 hours). The pH of culture medium can be maintained at a particular level via the addition of acids, bases, and/or buffering agents. In certain embodiments, culturing yeast at a pH of 6 or higher can reduce the production of unwanted side products such as olivetol. In some embodiments, the pH of the yeast culture ranges from about 6 to about 8. In some embodiments, the pH of the yeast culture is about 6.5. In some embodiments, the pH of the yeast culture is about 7. In some embodiments, the pH of the yeast culture is about 8.

In some embodiments, a recombinant yeast cell is genetically modified such that it produces, when cultured in vivo in a suitable precursor-containing media as described above, the cannabinoid product of interest or an intermediate at a level of at least about 0.1 g/L, at least about 0.5 g/L, at least about 0.75 g/L, at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 7 g/L, at least about 8 g/L, at least about 9 g/L, or at least 10 g/L. In some embodiments, a recombinant yeast cell is genetically modified such that it produces, when cultured in vivo in a suitable medium, the cannabinoid product of interest or an intermediate at a level of at least about 20 g/L, at least about 30 g/L, at least about 50 g/L, or at least about 80 g/L.

Cannabinoid production may be carried out in any vessel that permits cell growth and/or incubation. For example, a reaction mixture may be a bioreactor, a cell culture flask or plate, a multiwell plate (e.g., a 96, 384, 1056 well microtiter plates, etc.), a culture flask, a fermenter, or other vessel for cell growth or incubation. Biologically produced products of interest may be isolated from the fermentation medium or cell extract using methods known in the art. For example, solids or cell debris may be removed by centrifugation or filtration. Products of interest may be isolated, for example, by distillation, liquid-liquid extraction, membrane evaporation, adsorption, or other methods.

### Conversion of Cannabinoid Starting Materials and Intermediates to Cannabinoid Products

In some embodiments, the methods include expressing a cannabinoid precursor in a yeast cell, isolating the yeast cell, and converting the cannabinoid precursor to the cannabinoid product in the isolated yeast cell. In some embodiments, the cannabinoid precursor is olivetol, olivetolic acid, divarinol, or divarinic acid. Converting the cannabinoid precursor to the cannabinoid product can be conducted using the procedures described herein (e.g., chemical or enzymatic prenylation, thermal or enzymatic decarboxylation, etc.), or the conversion route can be modified according to the identity of the particular cannabinoid precursor or the particular cannabinoid product. Isolating the yeast cells can optionally include: collecting yeast cells from culture media by centrifugation, filtration, or other means; washing yeast cells to remove culture media or other components; removing at least a portion of liquid (e.g., culture media) from the cells; and/or drying the cells (e.g., by lyophilization or other means). Isolated yeast cells can be directly subjected to reaction conditions for forming the cannabinoid products. For example, yeast cells can be combined directly with solvents and other reagents.

In some embodiments, converting the 2-alkyl-4,6-dihydroxybenzoic acid or the 5-alkyl-benzene-1,3-diol to the cannabinoid product comprises producing a prenylated 2-alkyl-4,6-dihydroxybenzoic acid or a prenylated 5-alkyl-benzene-1,3-diol.

In the some embodiments, converting the cannabinoid precursor to the cannabinoid product may be conducted in vivo. In some embodiments, for example, the modified recombinant host cell further comprises fourth polynucleotide that encodes a prenyltransferase, and culturing the modified recombinant host cell comprises expressing the prenyltransferase encoded by the fourth exogenous polynucleotide and producing the prenylated 2-alkyl-4,6-dihydroxybenzoic acid or the prenylated 5-alkyl-benzene-1,3-diol in vivo.

Alternatively, the converting steps may be conducted in vitro. In the some embodiments, for example, producing a prenylated 2-alkyl-4,6-dihydroxybenzoic acid or prenylated 5-alkyl-benzene-1,3-diol comprises:
forming a reaction mixture comprising 1) the 2-alkyl-4,6-dihydroxybenzoic acid or the 5-alkyl-benzene-1,3-diol and 2) geraniol, an activated geraniol, or citral, and
maintaining the reaction mixture under conditions sufficient to form the prenylated 2-alkyl-4,6-dihydroxybenzoic acid or the prenylated 5-alkyl-benzene-1,3-diol.

In some embodiments, chemical prenylation may include forming a reaction mixture comprising (i) a 2-alkyl-4,6-dihydroxybenzoic acid (e.g., divarinic acid) or a 5-alkylbenzene-1,3-diol (e.g., divarinol), (ii) geraniol, an activated geraniol (e.g., geranyl bromide, geranyl chloride, geranyl tosylate, geranyl mesylate, or the like), or citral, and (iii) an organic solvent under conditions sufficient to produce an acidic cannabinoid (e.g., cannabigerovarinic acid, CBGVA) or a neutral cannabinoid (e.g., cannabigerovarin, CBGV). The method can be employed to convert divarinic acid analogs to the corresponding acidic cannabinoids, or to convert divarinol analogs to the corresponding neutral cannabinoids. The chemical prenylation may be conducted using conditions described, for example, in International Pat. Appl. No. PCT/US2020/066965, which is incorporated herein by reference in its entirety. The cannabinoid precursor may be present in a yeast mixture (e.g., dried yeast cells, or a wet yeast cell pellet collected from culture). In some such embodiments, the reaction mixture comprises the host cell (e.g., dried yeast cells). In some embodiments, the reaction mixture further comprises an acid. In some embodiments, the reaction mixture further comprises an amine (e.g., N,N-diisopropylethylamine, trimethylamine, pyridine, and diamines such as 1,2-diamines). In some embodiments, the reaction mixture includes citral and N,N-dimethylethylenediamine.

In some embodiments, an acidic cannabinoid such as CBGVA is the cannabinoid product. In some embodiments, the method further includes converting the acidic cannabinoid, e.g., CBGVA, to a neutral cannabinoid or another acidic cannabinoid as the cannabinoid product. In some embodiments, conversion of an intermediate compound such as CBGVA to another cannabinoid is carried out via physical or chemical processes such as heating, auto-oxidation or UV light treatment. For example, the methods can include the decarboxylation of acidic cannabinoid, either within the engineered yeast cells or following their full or partial purification through the action of heat or through the action of a wild-type or mutant decarboxylase enzyme contacting the cannabinoid acid in vivo or in vitro. Decarboxylation of the acidic cannabinoids provides corresponding neutral cannabinoids; decarboxylation of CBGA, for example, provides CBG.

In some embodiments, an acidic cannabinoid, e.g., CBGVA, may be decarboxylated to form a neutral cannabinoid compound, e.g., CBGVN, using a decarboxylase, e.g., Aspergillus nidulans orsB decarboxylase. Alternatively, an acidic cannabinoid can be decarboxylated by maintaining the acidic cannabinoid at an elevated temperature (e.g., around 40° C., 50° C., or 100° C.) for periods of time ranging from a few minutes to several hours.

In some embodiments, cannabinoid products set forth in Table 2 can be prepared using chemical steps and/or cannabinoid synthase-catalyzed steps, as described below. Examples of cannabinoid products include, but are not limited to, those described in WO 2020/092823.

TABLE 2
Cannabinoid Products

| Cannabinoid derivative | |
|---|---|
| structure | Derivative name |
| 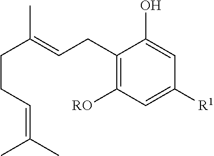 | cannabigerol [CBG] and analogs (R = H) cannabigerol monomethyl ether [CBGM] and analogs (R = CH₃) cannabigerovarin [CBGV] and analogs |
| 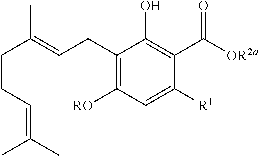 | cannabigerolic acid A [CBGA] and analogs (R = H) cannabigerolic acid A monomethyl ether [CBGAM] and analogs (R = CH₃) cannabigerovarinic acid [CBGVA] and analogs |
| 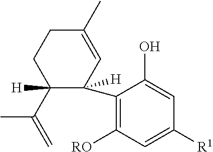 | (−)-cannabidiol [CBD] and analogs (R = H) cannabidiol monomethyl ether [CBDM] and analogs (R = CH₃) cannabidivarin [CBDV] and analogs cannabidiorcol [CBD-C1] and analogs |
| 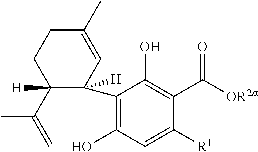 | cannabidiolic acid [CBDA] and analogs cannabidivarinic acid [CBDVA] and analogs |
| 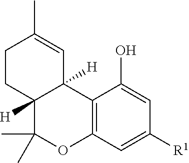 | Δ⁹-tetrahydrocannabinol [THC] and analogs Δ⁹-tetrahydrocannabivarin [THCV] and analogs Δ⁹-tetrahydrocannabiorcol [THC-C₁] and analogs |
| 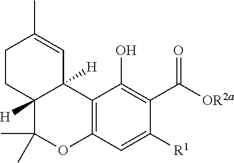 | Δ⁹-tetrahydrocannabinolic acid [Δ⁹-THCA] and analogs Δ⁹-tetrahydrocannabivarinic acid [Δ⁹-THCVA] and analogs Δ⁹-tetrahydrocannabiorcolic acid [THCOA] and analogs |
| 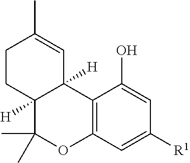 | (−)-(6aS, 10aR)-Δ⁹-tetrahydrocannabinol [cis-Δ⁹-THC] and analogs |
| 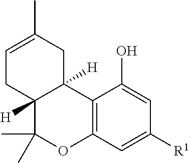 | (−)-Δ⁸-trans-(6aR,10aR)-Δ⁸-Δ⁸-tetrahydrocannabinol [Δ⁸-THC] and analogs (−)-Δ⁸-trans-(6aR,10aR)-Δ⁸-Δ⁸-tetrahydrocannabivarin [Δ⁸- THCV] and analogs |
| 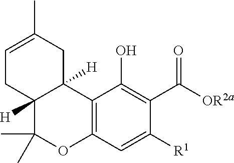 | (-)-Δ⁸-trans-(6aR,10aR)-Δ⁸-tetrahydrocannabinolic acid [Δ⁸- THCA] and analogs Δ⁸-tetrahydrocannabivarinic acid [Δ⁸-THCVA] and analogs |
| 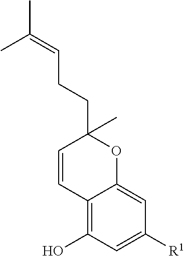 | cannabichromene [CBC] and analogs cannabichromevarin [CBCV] and analogs |
| 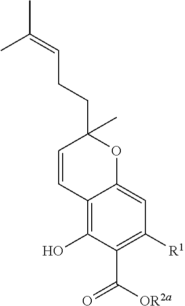 | cannabichromenic acid [CBCA] and analogs cannabichromevarinic acid [CBCVA] and analogs |
| 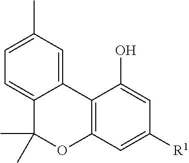 | cannabinol [CBN] and analogs cannabinol methyl ether [CBNM] and analogs cannabivarin [CBV] and analogs cannabiorcol [CBN-C1] and analogs |
| 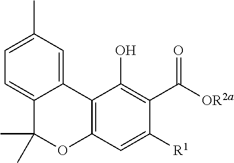 | cannabinolic acid [CBNA] and analogs cannabivarinic acid [CBVA] and analogs |
| 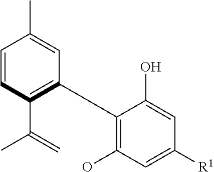 | cannabinodiol [CBND] and analogs cannabinodivarin [CBND-C3] and analogs |
| 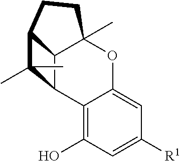 | (±)-(1aS,3aR,8bR,8cR)-cannabicyclol [CBL] and analogs (±)-(1aS,3aR,8bR,8cR)-cannabicyclovarin [CBLV] and analogs |
| 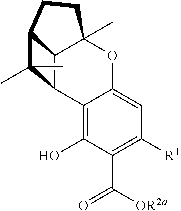 | (±)-(laS,3aR,8bR,8cR)-cannabicyclolic acid [CBLA] and analogs |
| 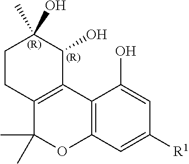 | (−)-(9R,10R)-trans-cannabitriol [(−)-trans-CBT] and analogs |
| 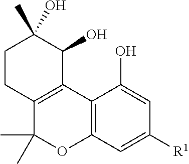 | (+)-(9S,10S)-trans-cannabitriol [(+)-trans-CBT] and analogs |
| 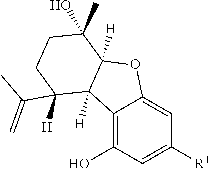 | (5aS,6S,9R,9aR)-cannabielsoin [CBE] and analogs |
| 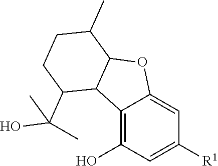 | cannabiglendol-C₃ [OH-iso-HHCV-C₃] and analogs |
| 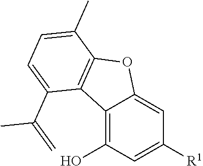 | dehydrocannabifuran [DCBF] and analogs |
| 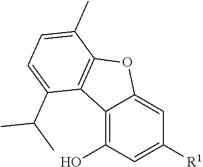 | cannabifuran [CBF] and analogs |
| 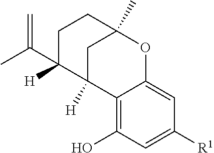 | (−)-Δ⁷-trans-(1R,3R,6R)-isotetrahydrocannabinol and analogs (−)-Δ⁷-trans-(1R,3R,6R)-isotetrahydrocannabivarin |
| 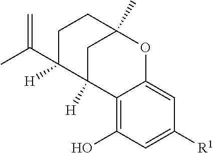 | (+)-Δ⁷-1,2-cis-(1R,3R,6S)-isotetrahydrocannabivarin and analogs |
| 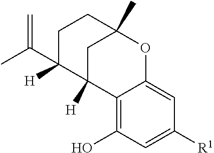 | (+)-Δ⁷-1,2-cis-(1S,3S,6R)-isotetrahydrocannabivarin and analogs |
| 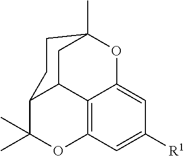 | cannabicitran [CBTN] and analogs |
| 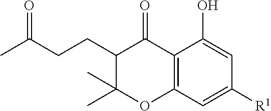 | cannabichromanone [CBCN] and analogs |
| 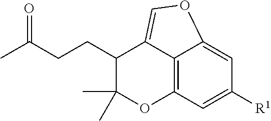 | cannabicoumaronone [CBCON] and analogs |

Cannabinoid products include, without limitation, CBG, CBDA, CBD, THC, Δ⁸-THC, THCA, Δ⁸-THCA, CBCA, CBC, CBN, CBND, CBNA, CBV, CBVA, THCV, THCVA, Δ⁸-THCA, CBGV, CBGVA, CBCV, CBCVA, CBDV, CBDVA, CBTN, as well as analogs thereof. Further examples include, but are not limited to, the cannabichromanones, cannabicoumaronone, 10-oxo-Δ^{6a(10a)}-tetrahydrohydrocannabinol (OTHC), cannabiglendol, and Δ⁷-isotetrahydrocannabinol.

### IV. EXAMPLES

### Example 1. Production of Divarinic Acid and Divarinol in Recombinant Yeast

An overnight culture of yeast cells expressing the C. sativa olivetolic acid PKS system (the C. sativa tetraketide synthase (TKS) and an engineered C. Sativa cyclase), and transformed with DNA constructs for the production of butanoyl-CoA using the Roseburia hominis butanoyl-CoA-transferase, was grown in 3 mL of Leu-, Ura-minimal media. 300 μL of the overnight culture was then inoculated into 3 mL culture tubes of YPD (2% D, 10 mM riboflavin and 50 μM pantothenic acid). The cells were grown overnight at 30° C. and 250 rpm. In the morning and evening, a 2 mM butanoic acid bolus was fed from a 1M ethanol solution and the culture was grown overnight. The next morning and evening, 2 mM butanoic acid was fed from a 0.3M butanoic acid stock diluted in ethanol, and the culture grown overnight. 2 mM butanoic acid feeds (0.3M stock in ethanol) were repeated for a third day. The culture was extracted, and divarinic acid and divarinol production were measured by HPLC at the 72-hour time point. The yield of divarinic acid was 628 mg/L, and the yield of divarinol was 93 mg/L. When this experiment was repeated in a 2-liter glucose-fed fermenter, the yield of divarinic acid was increased to around 1.4 g/L.

### Example 2. Production of Olivetolic Acid Analogs and Olivetol Analogs in Recombinant Yeast

Yeast cells expressing the C. sativa olivetolic acid PKS system were transformed with DNA constructs for various CoA-transferases or CoA-ligases and cultured as described in Example 1, feeding with a range of fatty acid substrates in addition to butanoic acid. As shown in Table 3, R. hominis butyryl-CoA:acetate CoA-transferase was found to be particularly useful in the production of a variety of olivetolic acid analogs and olivetol analogs. Notably, the yields of olivetolic acid and divarinic acid from cultures expressing R. hominis butyryl-CoA:acetate CoA-transferase were three-fold higher and twelve-fold higher, respectively, than from cultures expressing C. sativa AAE3.

TABLE 3
Production of olivetolic acid, olivetol and their analogs, including divarinic
acid and divarinol using selected acyl-CoA-transferases and ligases.

| | CsAAE3 ¹ | | revS ² | | Mig ³ | | FAA2 ⁴ | |
|---|---|---|---|---|---|---|---|---|
| | OA ^{a} | OL ^{b} | OA | OL | OA | OL | OA | OL |
| Fatty acid substrate | analog | analog | analog | analog | analog | analog | analog | analog |
| Octanoic acid | 4 | 9 | 5 | 8 | 4 | 4 | 2 | 1 |
| Heptanoic acid | 34 | 34 | 73 | 43 | 8 | 3 | 55 | 59 |
| Hexanoic acid | 133 | 72 | 119 | 66 | 10 | 2 | 231 | 102 |
| 6-Fluorohexanoic acid | 118 | 80 | 92 | 53 | 13 | 0 | 144 | 85 |
| 5-Chloropentanoic acid | 55 | 16 | 60 | 6 | 9 | 0 | 92 | 24 |
| Butanoic acid | 25 | 3 | 11 | 0 | 13 | 3 | 58 | 9 |
| 4-Fluorobutanoic acid | 53 | 19 | 0 | 0 | 29 | 8 | 7 | 1 |
| 3-Methylbutanoic acid | 0 | 0 | 3 | 0 | 6 | 0 | 0 | 0 |
| 2-Methylpropanoic acid | 0 | 0 | 0 | 0 | 24 | 36 | 0 | 0 |
| | A. th ⁵ | | FADK ⁶ | | R. ho ⁷ | | PCT ⁸ | |
| | OA | OL | OA | OL | OA | OL | OA | OL |
| Fatty acid substrate | analog | analog | analog | analog | analog | analog | analog | analog |
| Octanoic acid | 4 | 5 | 5 | 9 | 5 | 11 | 4 | 9 |
| Heptanoic acid | 41 | 45 | 60 | 55 | 58 | 50 | 53 | 49 |
| Hexanoic acid | 215 | 102 | 265 | 114 | 393 | 136 | n/d | n/d |
| 6-Fluorohexanoic acid | 100 | 64 | 79 | 93 | n/d | n/d | n/d | n/d |
| 5-Chloropentanoic acid | 58 | 12 | 24 | 9 | 153 | 35 | n/d | n/d |
| Butanoic acid | 42 | 5 | 12 | 0 | 314 | 51 | n/d | n/d |
| 4-Fluorobutanoic acid | 32 | 9 | 0 | 0 | 228 | 70 | 154 | 45 |
| 3-Methylbutanoic acid | 13 | 0 | 0 | 0 | 287 | 26 | n/d | n/d |
| 2-Methylpropanoic acid | 0 | 0 | 0 | 0 | 156 | 224 | 114 | 168 |

¹ C. sativa CsAAE3
² Streptomyces sp. SN-593 revS medium chain fatty acid acyl-CoA-ligase
³ M. avium mig medium chain Acyl-CoA-ligase
⁴ S. cerevisiae FAA2 medium chain acyl-CoA-ligase
⁵ A. thaliana AT4g05160 coumarate acyl-CoA-ligase
⁶ E. coli FADK acyl-CoA-ligase
⁷ R. hominis butyryl-CoA:acetate CoA-transferase
⁸ C. necator propionate-CoA-transferase
^{a} OA analog = olivetolic acid analog yield (mg/L)
^{b} OL analog = olivetol analog yield (mg/L)

### Example 3. Production of Cannabigerovarinic Acid (CBGVA)

Yeast cells transformed with a plasmid encoding GOT3 (amino acids 80-398) were grown as an overnight culture in 3 mL of Leu-minimal media. 500 μL of the overnight culture was then inoculated into 5-mL flasks of YPD (2% D, 10 mM riboflavin, 50 μM pantothenic acid, 1% Tween20). The cells were grown overnight at 30° C. and 250 rpm. In the morning and evening, 0.5 mM crude divarinic acid extract, prepared as described in Example 1 and dissolved in EtOH, was added (1 mM divarinic total). The cells were grown for an additional 48-72 hrs and CBGVA production was measured by HPLC. 148 mg/L of CBGVA was obtained.

### Example 4. Production of CBGA from Hexanoic acid and CBGVA from Butyric Acid in Engineered S. cerevisiae Expressing the Roseburia hominis Butyryl-CoA:acetate CoA-Transferase.

Yeast strain Y551 (URA+, LEU-) was engineered to express mevalonate pathway enzymes as described above for overproduction of geranyl-pyrophosphate (GPP), and to contain multiple copies of the geranylpyrophosphate:olivetolate geranyltransferase (GOT) (also referred to as cannabigerolic acid synthase, CBGAS) integrated into the yeast genome. The hSOD GOT DNA cassettes were integrated into the yeast genome at loci predicted to encode non-essential genes. The first URA-marked cassette contained a single hSOD-GOT gene driven by the S. cerevisiae ADH2 promoter and integrated at the YEL060C locus into strain Y407 to give strain Y523 after URA maker removal by counterselection on agar containing 5-fluororotic acid (FOA). The second URA marked cassette (A146) integrated at the YHR090C locus contained two bidirectionally arranged hSOD-GOT genes, each driven by a separate promoter (S. cerevisiae TEF1 and S. cerevisiae FBA1 promoters) to give strain Y531 after URA counterselection. The third URA marked cassette (A147) integrated at the YER024W locus contained two bidirectionally arranged hSOD-GOT genes, each driven by a separate promoter (S. cerevisiae TEF2 and S. cerevisiae TDH3 promoters) to give strain Y540 after URA counterselection. The fourth URA marked cassette (A150) contained the same hSOD-GOT and promoter configuration as that of A147 but was integrated at the YIL114C locus to give strain Y547 after URA counterselection. Finally, the fifth URA marked cassette (A151) contained the same hSOD-GOT and promoter configuration as that of A146 but was integrated at the YHR162W locus to give the URA prototrophic strain, Y551 containing a total of 9 integrated copies of hSOD-GOT.

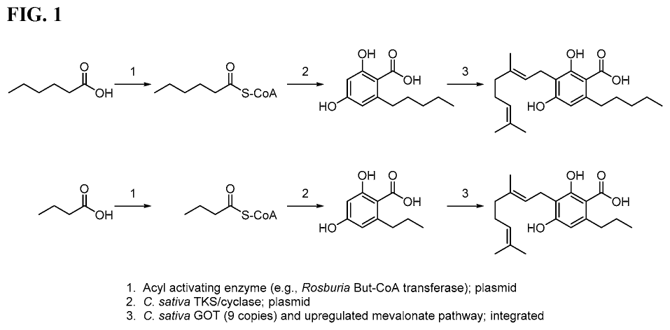

Plasmid pJK154L was transformed into Y551 with selection on minimal medium agar without leucine and without uracil. Plasmid pJK154L contains the C. sativa olivetol synthase gene (with a human superoxide dismutase [hSOD] fusion partner sequence) expressed from the S. cerevisiae ADH2 promoter (hSOD-TKS); the C. sativa olivetolic acid cyclase (cyclase; for cyclization of the tetraketide with retention of the acid) expressed from the S. cerevisiae TEF1 promoter; and the Roseburia hominis Butyryl-CoA:acetate CoA-transferase (But-CoA T), expressed from the S. cerevisiae FBA1 promoter (see, FIG. 1).

Primary transformants were “picked and patched” to fresh agar plates. Minimal liquid medium without uracil or leucine (3 mL in 15 mL culture tube) was inoculated with cells from the fresh patch. The culture was grown overnight in a shaker/incubator at 30° C. Rich medium (YP, 2% dextrose, 3 mL in a 15 mL culture tube) was inoculated with 300 μL of the overnight culture and grown overnight in a shaker/incubator at 30° C.

After overnight incubation (and when culture OD reached ˜8-10), hexanoic acid (1 mM final concentration) was added to a first culture and butyric acid (2 mM final concentration) was added to a second culture. Incubation of the cultures at 30° C. with shaking was continued. After 24 hours, second aliquots of hexanoic acid or butyric acid were added to the cultures, such that the final concentration acid was 2 mM for hexanoic acid or 4 mM for butyric acid. 24 hours after addition of the second acid aliquots (48 hours after the first addition), an aliquot (500 μL) of each culture was analyzed for the production of polyketides and cannabinoids. To a 500 μL aliquot of the whole cell broth from the hexanoic acid and butyric acid fed cultures was added 500 μL of isopropanol. The cell culture broth:isopropanol mix was vortexed for 30 seconds and the cellular debris was removed by centrifugation. Supernatant (20 μL) was analyzed by HPLC to determine the amounts of polyketides and cannabinoids produced. The contents of extracts from strains fed either hexanoic acid or butyric acid are summarized in Table 4.

TABLE 4
Mole fraction of polyketides and cannabinoids produced from
yeast cultured with hexanoic acid and butyric acid.

| Feed Acid | OL^{a} | DVL^{b} | OA^{c} | DVA^{d} | CBGA^{e} | CBGVA^{f} | CBFA^{g} |
|---|---|---|---|---|---|---|---|
| Hexanoic | 0.19 | 0.00 | 0.19 | 0.29 | 0.30 | 0.00 | 0.03 |
| Butyric | 0.00 | 0.24 | 0.00 | 0.75 | 0.00 | 0.02 | 0.00 |

^{a}olivetol;
^{b}divarinol;
^{c}olivetolic acid;
^{d}divarinic acid;
^{e}cannabigerolic acid;
^{f}cannabigerovarinic acid;
^{g}farnesylated olivetolic acid (sesqui-CBGA)

For the strain cultured with hexanoic acid, the major product was CBGA. The production of divarinic acid in this strain is believed to be due to the fact that the yeast strain produces some butyryl-CoA, either from endogenous production or from a precursor present in the medium. For the strain that was cultured with butyric acid, CBGVA was produced along with substantial amounts of divarinic acid.

### V. EXEMPLARY EMBODIMENTS

Exemplary embodiments provided in accordance with the presently disclosed subject matter include, but are not limited to, the claims and the following embodiments:
1. A modified recombinant host cell comprising a first exogenous polynucleotide that encodes a CoA-transferase that converts an aliphatic carboxylic acid to an acyl CoA thioester, wherein the modified recombinant host cell is engineered to produce a cannabinoid product.
2. The modified recombinant host cell of embodiment 1, wherein the CoA-transferase is selected from the group consisting of R. hominis butyryl-CoA:acetate CoA-transferase, E. coli acetyl-CoA:acetoacetyl CoA-transferase, and C. necator H16 propionate CoA-transferase.
3. A modified recombinant host cell comprising a first exogenous polynucleotide that encodes a CoA-ligase,
wherein the CoA-ligase is selected from the group consisting of M. avium mig medium chain acyl-CoA-ligase, and A. thaliana AT4g05160 coumarate acyl-CoA-ligase, and
wherein the modified recombinant host cell is engineered to produce a cannabinoid product.
4. The modified recombinant host cell of any one of embodiments 1-3, wherein the modified recombinant host cell further comprises one or more exogenous polynucleotides selected from the group consisting of:
a second exogenous polynucleotide that encodes a polyketide synthase (PKS), and
a third exogenous polynucleotide that encodes a 2-alkyl-4,6-dihydroxybenzoic acid cyclase.
5. The modified recombinant host cell of embodiment 4, wherein the PKS is a type I PKS, a type II PKS, or a type III PKS.
6. The modified recombinant host cell of embodiment 4 or embodiment 5, wherein the 2-alkyl-4,6-dihydroxybenzoic acid cyclase is olivetolic acid cyclase, an AtHS1 polypeptide, or the N-terminal domain of a BenH polypeptide.
7. The modified recombinant host cell of any one of embodiments 4-6, wherein the modified recombinant host cell further comprises a fourth polynucleotide that encodes a prenyltransferase.
8. The modified recombinant host cell of embodiment 7, wherein the prenyltransferase is geranylpyrophosphate:olivetolate geranyltransferase.
9. The modified recombinant host cell of embodiment 7 or embodiment 8, wherein multiple copies of the fourth polynucleotide are integrated into the host cell genome.
10. The modified recombinant host cell of any one of embodiments 1-9, wherein expression of one or more of the exogenous polynucleotides is driven by an ADH2 promoter, an ADH1 promoter, a GAL1 promoter, a MET25 promoter, a CUP1 promoter, a GPD promoter, a PGK promoter, a PYK promoter, a TPI promoter, a TEF1 promoter, or an FBA1 promoter.
11. The modified recombinant host cell of any one of embodiments 1-10, wherein at least two of the exogenous polynucleotides are present in the same autonomously replicating expression vector and expressed as a multicistronic mRNA.
12. The modified recombinant host cell of any one of embodiments 1-11, wherein the host cell is genetically modified to overexpress one or more mevalonate pathway enzymes.
13. The modified recombinant host cell of embodiment 12, wherein the mevalonate pathway enzymes are selected from the group consisting of erg10, erg13, thmgr, erg12, erg8, mvd1, idi1, erg20 F96WN127W, E. faecalis mvaE, and E. faecalis mvaS.
14. The modified recombinant host cell of any one of embodiments 1-13, wherein the modified recombinant host cell is a cell selected from the group consisting of Saccharomyces cerevisiae, Kluyveromyces lactis, Kluyveromyces marxianus, Pichia pastoris, Yarrowia lipolytica, Hansenula polymorpha and Aspergillus
15. A method of producing a cannabinoid product, the method comprising:
culturing a modified recombinant host cell comprising a first exogenous polynucleotide that encodes a CoA-transferase that converts an aliphatic carboxylic acid to an acyl CoA thioester under conditions in which the CoA-transferase encoded by the exogenous polynucleotide is expressed and the acyl CoA thioester is produced; and converting the acyl CoA thioester to the cannabinoid product.
16. The method of embodiment 15, wherein the CoA-transferase is selected from the group consisting of R. hominis butyryl-CoA:acetate CoA-transferase, E. coli acetyl-CoA:acetoacetyl-CoA-transferase, and C. necator H16 propionate CoA-transferase.
17. A method of producing a cannabinoid product, the method comprising:
culturing a modified recombinant host cell comprising a first exogenous polynucleotide that encodes a CoA-ligase that converts an aliphatic carboxylic acid to an acyl CoA thioester under conditions in which the CoA-ligase encoded by the exogenous polynucleotide is expressed and the acyl CoA thioester is produced; and
converting the acyl CoA thioester to the cannabinoid product;
wherein the CoA-ligase is selected from the group consisting of M. avium mig medium chain acyl-CoA-ligase and A. thaliana AT4g05160 coumarate acyl-CoA-ligase.
18. The method of any one of embodiments 15-17, wherein the aliphatic carboxylic acid is a C₂₋₅ carboxylic acid.
19. The method of any one of embodiments 15-17, wherein the aliphatic carboxylic acid is a C₆₋₂₀ carboxylic acid.
20. The method of any one of embodiments 15-19, wherein the aliphatic carboxylic acid comprises a carbon-carbon double bond, a hydroxy group, a halogen, deuterium, tritium, or a combination thereof.
21. The method of any one of embodiments 15-20, wherein the modified recombinant host cell further comprises one or more exogenous polynucleotides selected from the group consisting of:
a second exogenous polynucleotide that encodes a polyketide synthase (PKS) that produces a polyketide from the acyl CoA thioester and malonyl CoA, and
a third exogenous polynucleotide that encodes a 2-alkyl-4,6-dihydroxybenzoic acid cyclase;
wherein culturing the modified recombinant host cell comprises expressing products encoded by the second and third exogenous polynucleotides and converting the acyl CoA thioester to a 2-alkyl-4,6-dihydroxybenzoic acid or a 5-alkyl-benzene-1,3-diol; and
wherein converting the acyl CoA thioester to the cannabinoid product comprises converting the 2-alkyl-4,6-dihydroxybenzoic acid or the 5-alkyl-benzene-1,3-diol to the cannabinoid product.
22. The method of embodiment 21, wherein the 2-alkyl-4,6-dihydroxybenzoic acid is divarinic acid.
23. The method of embodiment 21 or embodiment 22, wherein the PKS is a type I PKS, a type II PKS, or a type III PKS.
24. The method of any one of embodiments 21-23, wherein the 2-alkyl-4,6-dihydroxybenzoic acid cyclase is olivetolic acid cyclase, an AtHS1 polypeptide, or the N-terminal domain of a BenH polypeptide.
25. The method of any one of embodiments 21-24, wherein converting the 2-alkyl-4,6-dihydroxybenzoic acid or the 5-alkyl-benzene-1,3-diol to the cannabinoid product comprises producing a prenylated 2-alkyl-4,6-dihydroxybenzoic acid or a prenylated 5-alkyl-benzene-1,3-diol.
26. The method of embodiment 25, wherein the modified recombinant host cell further comprises fourth polynucleotide that encodes a prenyltransferase, and wherein culturing the modified recombinant host cell comprises expressing the prenyltransferase encoded by the fourth exogenous polynucleotides and producing the prenylated 2-alkyl-4,6-dihydroxybenzoic acid or the prenylated 5-alkyl-benzene-1,3-diol.
27. The method of embodiment 26, wherein the prenyltransferase is geranylpyrophosphate:olivetolate geranyltransferase.
28. The method of embodiment 26 or embodiment 27, wherein multiple copies of the fourth polynucleotide are integrated into the host cell genome.
29. The method of embodiment 25, wherein producing the prenylated 2-alkyl-4,6-dihydroxybenzoic acid or the prenylated 5-alkyl-benzene-1,3-diol comprises:
forming a reaction mixture comprising 1) the 2-alkyl-4,6-dihydroxybenzoic acid or the 5-alkyl-benzene-1,3-diol and 2) geraniol, an activated geraniol, or citral, and
maintaining the reaction mixture under conditions sufficient to form the prenylated 2-alkyl-4,6-dihydroxybenzoic acid or the prenylated 5-alkyl-benzene-1,3-diol.
30. The method of embodiment 29, wherein the reaction mixture further comprises a diamine.
31. The method of any one of embodiments 15-30, wherein expression of one or more of the exogenous polynucleotides is driven by an ADH2 promoter, an ADH1 promoter, a GAL1 promoter, a MET25 promoter, a CUP1 promoter, a GPD promoter, a PGK promoter, a PYK promoter, a TPI promoter, a TEF1 promoter, or an FBA1 promoter.
32. The method of any one of embodiments 15-31, wherein at least two of the exogenous polynucleotides are present in the same autonomously replicating expression vector and expressed as a multicistronic mRNA.
33. The method of any one of embodiments 15-32, wherein the host cell is genetically modified to overexpress one or more mevalonate pathway enzymes.
34. The method of embodiment 33, wherein the mevalonate pathway enzymes are selected from the group consisting of erg10, erg13, thmgr, erg12, erg8, mvd1, idi1, erg20 F96WN127W, E. faecalis mvaE, and E. faecalis mvaS.
35. The method of any one of embodiments 15-34, wherein the modified recombinant host cell is a cell selected from the group consisting of Saccharomyces cerevisiae, Kluyveromyces lactis, Kluyveromyces marxianus, Pichia pastoris, Yarrowia hpolytica, Hansenula polymorpha and Aspergillus.

The invention illustratively described herein suitably may be practiced in the absence of any element or elements, limitation or limitations that are not specifically disclosed herein. Thus, for example, in each instance herein any of the terms “comprising”, “consisting essentially of” and “consisting of” may be replaced with either of the other two terms. Thus, for example, some embodiments may encompass a host cell “comprising” a number of components, other embodiments would encompass a host cell “consisting essentially of” the same components, and still other embodiments would encompass a host cell “consisting of” the same components. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. In addition, each reference provided herein is incorporated by reference in its entirety to the same extent as if each reference was individually incorporated by reference.

ILLUSTRATIVE SEQUENCES
R. hominis butyryl-CoA: acetate
CoA-transferase
SEQ ID NO: 1
MDFREEYKQKLVSADEAVKLIKSGDWVDYGWCTNTVDALD
QALAKRTDELTDVKLRGGILMKPLAVFAREDAGEHFCWNS
WHMSGIERKMINRGVAYYCPIRYSELPRYYRELDCPDDVA
MFQVAPMDAHGYFNFGPSASHLGAMCERAKHIIVEVNENM
PRCLGGTECGIHISDVTYIVEGSNPPIGELGAGGPATDVD
KAVAKLIVDEIPNGACLQLGIGGMPNAVGSLIAESDLKDL
GVHTEMYVDAFVDIAKAGKINGSKKNIDRYRQTYAFGAGT
KKMYDYLDDNPELMSAPVDYTNDIRSISALDNFISINNAV
DIDLYGQVNAESAGIKQISGAGGQLDFVLGAYLSKGGKSF
ICLSSTFKTKDGQVQSRIRPTLANGSIVTDARPNTHYVVT
EYGKVNLKGLSTWQRAEALISIAHPDFRDDLIKEAEQMHI
WRRSNR
E. coli acetyl-CoA: acetoacetyl-CoA
transferase AtoA
SEQ ID NO: 2
MDAKQRIARRVAQELRDGDIVNLGIGLPTMVANYLPEGIH
ITLQSENGFLGLGPVTTAHPDLVNAGGQPCGVLPGAAMFD
SAMSFALIRGGHIDACVLGGLQVDEEANLANWVVPGKMVP
GMGGAMDLVTGSRKVIIAMEHCAKDGSAKILRRCTMPLTA
QHAVHMLVTELAVFRFIDGKMWLTEIADGCDLATVRAKTE
ARFEVAADLNTQRGDL
E. coli acetyl-CoA: acetoacetyl-CoA
transferase AtoD
SEQ ID NO: 3
MKTKLMTLQDATGFFRDGMTIMVGGFMGIGTPSRLVEALL
ESGVRDLTLIANDTAFVDTGIGPLIVNGRVRKVIASHIGT
NPETGRRMISGEMDVVLVPQGTLIEQIRCGGAGLGGFLTP
TGVGTVVEEGKQTLTLDGKTWLLERPLRADLALIRAHRCD
TLGNLTYQLSARNFNPLIAL
C. necator H16 propionate CoA-transferase
SEQ ID NO: 4
MKVITAREAAALVQDGWTVASAGFVGAGHAEAVTEALEQR
FLQSGLPRDLTLVYSAGQGDRGARGVNHFGNAGMTASIVG
GHWRSATRLATLAMAEQCEGYNLPQGVLTHLYRAIAGGKP
GVMTKIGLHTFVDPRTAQDARYHGGAVNERARQAIAEGKA
CWVDAVDFRGDEYLFYPSFPIHCALIRCTAADARGNLSTH
REAFHHELLAMAQAAHNSGGIVIAQVESLVDHHEILQAIH
VPGILVDYVVVCDNPANHQMTFAESYNPAYVTPWQGEAAV
AEAEAAPVAAGPLDARTIVQRRAVMELARRAPRVVNLGVG
MPAAVGMLAHQAGLDGFTLTVEAGPIGGTPADGLSFGASA
YPEAVVDQPAQFDFYEGGGIDLAILGLAELDGHGNVNVSK
FGEGEGASIAGVGGFINITQSARAVVFMGTLTAGGLEVRA
GDGGLQIVREGRVKKIVPEVSHLSFNGPYVASLGIPVLYI
TERAVFEMRAGADGEARLTLVEIAPGVDLQRDVLDQCSTP
IAVAQDLREMDARLFQAGPLHL
M. avium mig medium chain acyl-CoA-ligase
SEQ ID NO: 5
MSDTTTAFTVPAVAKAVAAAIPDRELIIQGDRRYSYRQVI
ERSNRLAAYLHSQGLGCHTEREALAGHEVGQDLLGLYAYN
GNEFVEALLGAFAARVAPFNVNFRYVKSELHYLLADSEAT
ALIYHAAFAPRVAEILPDLPRLRVLIQIADESGNELLDGA
VDYEDALASVSAEPPPVRHCPDDLYVLYTGGTTGMPKGVL
WRQHDIFMTSFGGRNLMTGEPSSSIDEIVQRAASGPGTKL
MILPPLIHGAAQWSVMTAITTGQTVVFPTVVDHLDAEDVV
RTIEREKVMVVTVVGDAMARPLVAAIEKGIADVSSLAVVA
NGGALLTPFVKQRLIEVLPNAVVVDGVGSSETGAQMHHMS
TPGAVATGTFNAGPDTFVAAEDLSAILPPGHEGMGWLAQR
GYVPLGYKGDAAKTAKTFPVIDGVRYAVPGDRARHHADGH
IELLGRDSVCINSGGEKIFVEEVETAIASHPAVADVVVAG
RPSERWGQEVVAVVALSDGAAVDAGELIAHASNSLARYKL
PKAIVFRPVIERSPSGKADYRWAREQAVDG
A. thaliana AT4g05160 coumarate
acyl-CoA-ligase
SEQ ID NO: 6
MEKSGYGRDGIYRSLRPTLVLPKDPNTSLVSFLFRNSSSY
PSKLAIADSDTGDSLTFSQLKSAVARLAHGFHRLGIRKND
VVLIFAPNSYQFPLCFLAVTAIGGVFTTANPLYTVNEVSK
QIKDSNPKIIISVNQLFDKIKGFDLPVVLLGSKDTVEIPP
GSNSKILSFDNVMELSEPVSEYPFVEIKQSDTAALLYSSG
TTGTSKGVELTHGNFIAASLMVTMDQDLMGEYHGVFLCFL
PMFHVFGLAVITYSQLQRGNALVSMARFELELVLKNIEKF
RVTHLWVVPPVFLALSKQSIVKKFDLSSLKYIGSGAAPLG
KDLMEECGRNIPNVLLMQGYGMTETCGIVSVEDPRLGKRN
SGSAGMLAPGVEAQIVSVETGKSQPPNQQGEIWVRGPNMM
KGYLNNPQATKETIDKKSWVHTGDLGYFNEDGNLYVVDRI
KELIKYKGFQVAPAELEGLLVSHPDILDAVVIPFPDEEAG
EVPIAFVVRSPNSSITEQDIQKFIAKQVAPYKRLRRVSFI
SLVPKSAAGKILRRELVQQVRSKM
S. cerevisiae FAA2 medium chain
acyl-CoA-ligase
SEQ ID NO: 7
MAAPDYALTDLIESDPRFESLKTRLAGYTKGSDEYIEELY
SQLPLTSYPRYKTFLKKQAVAISNPDNEAGFSSIYRSSLS
SENLVSCVDKNLRTAYDHFMFSARRWPQRDCLGSRPIDKA
TGTWEETFRFESYSTVSKRCHNIGSGILSLVNTKRKRPLE
ANDFVVAILSHNNPEWILTDLACQAYSLTNTALYETLGPN
TSEYILNLTEAPILIFAKSNMYHVLKMVPDMKFVNTLVCM
DELTHDELRMLNESLLPVKCNSLNEKITFFSLEQVEQVGC
FNKIPAIPPTPDSLYTISFTSGTTGLPKGVEMSHRNIASG
IAFAFSTFRIPPDKRNQQLYDMCFLPLAHIFERMVIAYDL
AIGFGIGFLHKPDPTVLVEDLKILKPYAVALVPRILTRFE
AGIKNALDKSTVQRNVANTILDSKSARFTARGGPDKSIMN
FLVYHRVLIDKIRDSLGLSNNSFIITGSAPISKDTLLFLR
SALDIGIRQGYGLTETFAGVCLSEPFEKDVGSCGAIGISA
ECRLKSVPEMGYHADKDLKGELQIRGPQVFERYFKNPNET
SKAVDQDGWFSTGDVAFIDGKGRISVIDRVKNFFKLAHGE
YIAPEKIENIYLSSCPYITQIFVFGDPLKTFLVGIVGVDV
DAAQPILAAKHPEVKTWTKEVLVENLNRNKKLRKEFLNKI
NKCTDGLQGFEKLHNIKVGLEPLTLEDDVVTPTFKIKRAK
ASKFFKDTLDQLYAEGSLVKTEKL
E. coli FADK acyl-CoA-ligase
SEQ ID NO: 8
MHPTGPHLGPDVLFRESNMKVTLTFNEQRRAAYRQQGLWG
DASLADYWQQTARAMPDKIAVVDNHGASYTYSALDHAASC
LANWMLAKGIESGDRIAFQLPGWCEFTVIYLACLKIGAVS
VPLLPSWREAELVWVLNKCQAKMFFAPTLFKQTRPVDLIL
PLQNQLPQLQQIVGVDKLAPATSSLSLSQIIADNTSLTTA
ITTHGDELAAVLFTSGTEGLPKGVMLTHNNILASERAYCA
RLNLTWQDVFMMPAPLGHATGFLHGVTAPFLIGARSVLLD
IFTPDACLALLEQQRCTCMLGATPFVYDLLNVLEKQPADL
SALRFFLCGGTTIPKKVARECQQRGIKLLSVYGSTESSPH
AVVNLDDPLSRFMHTDGYAAAGVEIKVVDDARKTLPPGCE
GEEASRGPNVFMGYFDEPELTARALDEEGWYYSGDLCRMD
EAGYIKITGRKKDIIVRGGENISSREVEDILLQHPKIHDA
CVVAMSDERLGERSCAYVVLKAPHHSLSLEEVVAFFSRKR
VAKYKYPEHIVVIEKLPRTTSGKIQKFLLRKDIMRRLTQD
VCEEIE
Ralstonia solanacearum MicC. In some
embodiments, the MicC amino acid
sequence comprises a Y1991A amino acid
substitution (Y1991 is underlined in
SEQ ID NO: 9).
SEQ ID NO: 9
MTTHALTERATLVDWIEHHARARPLAEALFFCGHGADDLR
LGYGALSERVRRCAAALQQRGAAGSTALILFPSGIDYVVA
LLACFYAGVTGVPVNLPGVSRVRRVLPKLGDITRDCRPAV
VLTHTAIERASGNDLRDFAAGHGLDILHLDTLGGEAAAWV
RPALTPESIAFLQYTSGSTGSPKGVVNRHGALLRNLQFLG
RLTRPQDRAPEDTAVASWLPLFHDLGLIMGILLPLAYGNR
AVYMAPMAFVADPLRWLEIATAERATALPCPSFALRLCAD
EARRAAPARTAGIDLSSVQCLMPAAEPVLPSQIEAFQAAF
AAHGMRREAIRPAYGLAEATLLVSANVDDAPPHRIDVETA
PLEQGRAVVHPAAAPMPAAGRRRYVSNGREFDGQDVRIVD
PRTCATLPEGTVGEIWISGPCIAGGYWNKAELNREIFMAE
TPGAGDRRYLRTGDMGFLHGGHLFVTGRLKDMMLFRGQCH
YPNDIEATSGRAHAAAIPESGAAFSIQAEDEAGERLVIVQ
EVRKQAGIDPRDIATAVRAAVAEGHALGVHAVVLIRKGTL
PRTTSGKVRRAAVREAWLAGTLQTLWQDDIDNLAVPPTPA
QETAAAPADAALLAALAPLDAARRQQHLVQWLAARAAAAL
GTVAARAIRPEASLFGYGLDSMSATRLAAVAAAASGLALP
DSLLFDHPSLDGLAGWLLQAMEQARHLPPAPGGRDRAMPA
PRPAAHRHGDGQDPIAIIGMAFRLPGENGHDADTDAAFWR
LLDGAGCAIRPMPAERFRAPAGMPGFGAYLNQVDRFDAAF
FGMSPREAMNTDPQQRLLLEVAWHALEDAGLPPGDLRGSD
SGVFVGIGTADYGHLPFISGDDAHFDAYWGTGTSFAAACG
RLSFTFGWEGPSMAVDTACSASHSALHLAVQALRARECGM
ALSAGVKLQLLPEIDRVLHKAGMLAADGRCKTLDASADGY
VRGEGCVVLVLKRLSDALADGDAIRAVIRDTLVRQDGAGS
SLSAPNGEAQQRLLSLALARAGLAPSEIDYIELHGTGTRL
GDPIEYQSVADVFGGRAPDDPLWIGSVKTNIGHLESAAGA
AGLVKTVLALEQARIPPLVGLKGINPLIDLDAIPARAPAH
TVDWPARQAVRRAGVTSYGFAGTIAHVILEQAPQAPVAQA
AGTEPTRGPHLFLLSARSPDALRRLAAAYRDTLAGTADLA
VLANGMARQREHHALRAAVVASDHDECARALDRLAAPDAA
APEAVTRAPRVGFLFTGQGSQYAGMTRALYAAQPDFRAAL
DAADAALAPHLGRSILALMHDDAQRDALQQTAHAQPALFA
CGYALAAMWQAWGVVPAVLVGHSIGEFAAMVVAGAMTLED
AARLIVRRGALMQALPAGGAMLAARATPRHAHDLLAALAP
AVAAEVSLAAINGPQDVVFSGSAAGIDAVRARLDAQQLDA
RPLAVSHAFHSPLLDPMLGDWAEACADAQSAPPRIPLIST
LTGAPMTTAPDAAYWSAHARQPVRFAEALARAGADCDVLL
EIGAHAVLSALAQRNQLAQPWPHPVACVASLLRGTDDSRA
VAQACAELYLRGQPFDWDRLFAGPLPSPRALPRYPFDRQS
HWLEYDEDAPRTPLPMQPQPERAAPRPVERYAVQWEPFAP
SAGDGHASTYWIVAADAADAGPADAGRLAARLSGPARDVH
VLSPSQWADAADRIADDDVVIYLAGWPARASDAAAVAGSR
HVWQLTECVRTLQRLRKTPRILLPTLHGQSPDGAPCDPLQ
AALWGAARPLSLEYPGPAWLLADCAGESPLETLADALPAL
LPLFGKEEAVALRAGGWLRPRLTPQAAPERAPCVTLRADG
LYLVAGAYGALGRHTTDWLAAHGATHLVLAGRRAPPAGWQ
ARLALLRAQGVRIDPVDADLAEAADVERLFDAVAALEATT
GRTLAGVFHCAGTSRENDLAGLTTDDCAAVTGAKMTGAWL
LHEQTRARRLDWFVCFTSISGVWGSRLQIPYGAANAFQDA
LVRLRRAQGLPALAVAWGPWGGGAGMSEVDDALLQLLRAA
GIRRLAPSRYLATLDHLLGHAEHADGLPADGTCVVAEVDW
QQFIPLFALYNPIGTFERCRTDTATHATAAPSALIALDSG
ARADAVRAFVIAELARTLRVAPSQLTPDIELLKLGMDSIL
VMDFSRRCESGLGVKCELKAIFERNTPGGLASYLLERLEH
APQGAVPAPAAAEPIVHAPDHAHLPFPLTELQHAYWIGRQ
GHYALGGVACHAYLEADAADGLDLGLLERCWNALVARHGA
LRLVIDESGQQRILPRVPAYRIRVANLGAATPQALAAHCD
DWRQAMSHQVLDAAQWPLFDVRATHLPGGATRLHIGIDML
INDATSGQIIWDELAALYRAGGDLERAGLAPFEISFRDYV
LAKYVHSEARRAARESAKAYWLGQLETLPPAPQLPLRAEA
LHRAAPRFSRRQHRLSAPQWQSLRDRAAASGCTPASLLIA
VFAEVLSAWSTEPRFTLNLTTFDRLPWHADVPRLLGDFTA
VTLLPLDCAAPLPFGQRAAAVNGAVLEHLQHRAFSAVDVL
REWNRGRERQDAVSMPVVFTSQLGMSDPTKGAARASVLGT
VGYGISQTPQVWLDHQACELDGALIYNWDAVDALFQPGVL
DAMFDAYNRMLERLAADADAWLEPLPALLPQAQREVRARV
NASTAPLPERCLDQLFFDQA
Truncated Ralstonia solanacearum MicA
SEQ ID NO: 10
MMTITTDRTPPAAGAALDRNRSAYAGLADVLERAGLAEHA
LYLNWGYRPVDGQPDWAARELPPGELGRMQARLVLEVLGD
TPLDGRRVLDVGCGRGGALALMGRLHAPAALAGADISAAN
IAYCRKRHTHPRLRFQIADACRLPYPDSSMDVVFNLESSG
AYPDIGAFFHHVHRILRVGGRFCLADVFDADSVAWVRAAL
EQAGFTLERERSIPAQVRAARERASPGIWRRLDTALTALD
APGLRRELERYLAAPSSGLFQALEDGRVDYRLFHWRKTCP
AAGRIDADVIARLATRSARLDAALQDRAPSAAAPQSPAPG
PANASASAWFPFTAPDAQAGFNVFALPYAGGGASVYRAWT
LPRRPGAAPWQLCPVQLPGRESRFGEPLIDDMATLADRLA
DAIGPYAHRPWALLGCSLGCKIAFEVARRFARQGRPPALL
FLMACPAPGLPLGRRISTRAEADFAREVCHLGGTPPEVLA
DAEMMRTLMPILRNDSALAEHYVAAEDATVNVPIVMVAAG
DDHLVTVEEARRWQRHAGAGFDWRLVDGGHFFLRQRRREL
TDWLLDALRRGERTLPVQTTTTDVPDVPCSTPEQPRDPSR
MPAPGASANLVLAPGEILVVTAPRSLAARLTPAVLSDDEQ
RQLARFAFDADRERYLAAHWAKRRVLGALLAAAPRSLRFG
AQAGGKPYLIGEALHFSLSHSGDRVAVAVCRHAPVGVDIE
QARGIACHASAARIMHPLDRIAPQCETPEDRFLAAWSLKE
AVAKCTGAGLALPFDSLRLAFAGNGRYGCLLGTHAAWEAH
HQHEDGVHLAVASATPWAALRILPLDAALAEG
Streptomyces sp. A2991200 BenA
(without signal peptide sequence from
amino acids 2-29 encoded by the
Streptomyces BenA gene)
SEQ ID NO: 11
MAGRTATRRITLFDPERFRCRIAAECDFDAAALGLTPQEI
RRMDRAVQMAVAATGEALADAGVGEGDLDPARTGVTIGNA
VGSTMMMEEEYVVISDGGRKWLCDEEYGVRHLYGAVIPST
AGVEVARRVGAEGPTAVVSTGCTSGLDAVGHAAQLIEEGS
ADVVIGGATDAPISPITVACFDSLKATSTRNDDAEHACRP
FDRDRDGLVLGEGSAVFVMEARERAVRRGAKIYCEVAGYA
GRANAYHMTGLKPDGRELAEAIDRAMAQAGISAEDIDYVN
AHGSGTRQNDRHETAAFKRSLRDHARRVPVSSIKSMVGHS
LGAIGAIEVAASALAIEHGVVPPTANLTTPDPECDLDYVP
REAREHPTDVVLSVGSGFGGFQSAVVLISPRSRR
Streptomyces sp. A2991200 BenQ
SEQ ID NO: 12
MSQLSLSQAAPAGGSRIRGVGAYRPARVVTNEEIAPRIGV
APEWIARRSGIHTRRFAGPDEPLAMMAATASEKALAAAGL
SADEVDCVLVATISHLLQMPALAVDVAHRLGAAPTAAFDL
SAACAGFCHGVAIADSMVRSGTAHNVLLVGADRMTDVVDA
DDPATAFLFADGAGAVVIGPSETPGIGPVAWGSDGERMDA
ITMTGHWTPSLRTNPELPWPYLCMTGWKVFRWATETMGQA
ARDAIERAGVTSEELSAFIPHQANGLITDALAKDIGLTAD
TAIARDITDSGNTSGASIPMAMERLLASGQARSGEAALLI
GFGSGLVHAGQVVLLP
BenB
SEQ ID NO: 13
MTVITGLGVVAPTGVGLDDYWATTLAGKSGIDRIRRFDPS
GYTAQLAGQVDDFEATDHVPSKLLAQTDRMTHFAFAGANM
ALADAHVDLADFPEYERAVVTANSSGGVEYGQHELQKMWS
GGPMRVSAYMSVAWFYAATTGQLSIHHGLRGPCGLIATEQ
AGGLDALGHARRLLRRGARIAVTGGTDAPLSPASMVAQLA
TGLLSSNPDPTAAYLPFDDRAAGYVPGEGGAIMIMEPAEH
ALRRGAERIYGEIAGYAATFDPAPGTGRGPTLGRAIRNAL
DDARIAPSEVDLVFADGSGTPAMDRAEAEALTEVFGPRGV
PVTVPKAATGRMYSGGGALDVATALLAMRDGVAPPTPHVT
ELASDCPLDLVRTEPRELPIRHALVCARGVGGFNAALVLR
RGDLTTPEH
BenC
SEQ ID NO: 14
MSTLSVEKLLEIMRATQGESADTSGLTEDVLDKPFTDLNV
DSLAVLEVVTQIQDEFKLRIPDSAMEGMETPRQVLDYVNE
RLEEAA
C. sativa olivetolic acid synthase
SEQ ID NO: 15
MNHLRAEGPASVLAIGTANPENILLQDEFPDYYFRVTKSE
HMTQLKEKFRKICDKSMIRKRNCFLNEEHLKQNPRLVEHE
MQTLDARQDMLVVEVPKLGKDACAKAIKEWGQPKSKITHL
IFTSASTTDMPGADYHCAKLLGLSPSVKRVMMYQLGCYGG
GTVLRIAKDIAENNKGARVLAVCCDIMACLFRGPSESDLE
LLVGQAIFGDGAAAVIVGAEPDESVGERPIFELVSTGQTI
LPNSEGTIGGHIREAGLIFDLHKDVPMLISNNIEKCLIEA
FTPIGISDWNSIFWITHPGGKAILDKVEEKLHLKSDKFVD
SRHVLSEHGNMSSSTVLFVMDELRKRSLEEGKSTTGDGFE
WGVLFGFGPGLTVERVVVRSVPIKY
C. sativa olivetolic acid cyclase
SEQ ID NO: 16
MAVKHLIVLKFKDEITEAQKEEFFKTYVNLVNIIPAMKDV
YWGKDVTQKNKEEGYTHIVEVTFESVETIQDYIIHPAHVG
FGDVYRSFWEKLLIFDYTPRK
Olivetolic acid cyclase polypeptide
sequence lacking the N-terminal
methionine and C-terminal lysine
relative to SEQ ID NO: 16
SEQ ID NO: 17
AVKHLIVLKFKDEITEAQKEEFFKTYVNLVNIIPAMKDVY
WGKDVTQKNKEEGYTHIVEVTFESVETIQDYIIHPAHVGF
GDVYRSFWEKLLIFDYTPR
Truncated version of cyclase, 95 aa,
lacking the N-terminal methionine and
five amino acid sequence YTPRK at the
C-terminal end relative to SEQ ID NO: 16
SEQ ID NO: 18
AVKHLIVLKFKDEITEAQKEEFFKTYVNLVNIIPAMKDVY
WGKDVTQKNKEEGYTHIVEVTFESVETIQDYIIHPAHVGF
GDVYRSFWEKLLIFD
Arabidopsis thaliana AtHS1 cyclase
SEQ ID NO: 19
MEEAKGPVKH VLLASFKDGV SPEKIEELIK
GYANLVNLIE PMKAFHWGKD VSIENLHQGY
THIFESTFES KEAVAEYIAH PAHVEFATIF
LGSLDKVLVI DYKPTSVSL
N-terminal domain of BenH polypeptide
from Streptomyces sp. A2991200
SEQ ID NO: 20
AGRTDNSVVIDAPVQLVWDMTNDVSQWAVLFEEYAESEVL
AVDGDTVRFRLTTQPDEDGKQWSWVSERTRDLENRTVTAR
RLDNGLFEYMNIRWEYTEGPDGVRMRWIQEFSMKPSAPVD
DSGAEDHLNRQTVKEMARIKKLIEEA
C. sativa GOT truncated sequence
(80-398 of the mature protein)
SEQ ID NO: 21
MSDQIEGSPHHESDNSIATKILNFGHTCWKLQRPYVVKGM
ISIACGLFGRELFNNRHLFSWGLMWKAFFALVPILSFNFF
AAIMNQIYDVDIDRINKPDLPLVSGEMSIETAWILSIIVA
LTGLIVTIKLKSAPLFVFIYIFGIFAGFAYSVPPIRWKQY
PFTNFLITISSHVGLAFTSYSATTSALGLPFVWRPAFSFI
IAFMTVMGMTIAFAKDISDIEGDAKYGVSTVATKLGARNM
TFVVSGVLLLNYLVSISIGIIWPQVFKSNIMILSHAILAF
CLIFQTRELALANYASAPSRQFFEFIWLLYYAEYFVYVFI
hSOD-GOT3 (hSOD sequence is underlined)
SEQ ID NO: 22
MATKAVCVLKGDGPVQGIINFEQKESNGPVKVWGSIKGLT
EGLHGFHVHEFGDNTAGCTSAGPHFNPLSRKHGGPKDEER
HVGDLGNVTADKDGVADVSIEDSVISLSGDHCIIGRTLVV
HEKADDLGKGGNEESTKTGNAGSRLACGVIGIAQPRSDQI
EGSPHHESDNSIATKILNFGHTCWKLQRPYVVKGMISIAC
GLFGRELFNNRHLFSWGLMWKAFFALVPILSFNFFAAIMN
QIYDVDIDRINKPDLPLVSGEMSIETAWILSIIVALTGLI
VTIKLKSAPLFVFIYIFGIFAGFAYSVPPIRWKQYPFTNF
LITISSHVGLAFTSYSATTSALGLPFVWRPAFSFIIAFMT
VMGMTIAFAKDISDIEGDAKYGVSTVATKLGARNMTFVVS
GVLLLNYLVSISIGIIWPQVFKSNIMILSHAILAFCLIFQ
TRELALANYASAPSRQFFEFIWLLYYAEYFVYVFI
Protein sequence translation from hops
CBDAS homolog nucleotide
sequence HL.Tea.v1.0.G019551
SEQ ID NO: 23
MNFRFSSPSLPKPSVIITPFHVSQIKATLMCSKKHGLQIR
TRSGGHDSDGLSYISDVPYVVIDLRNLSKVKVDVHDKTAW
VQAGATIGEVYYNIAKKSPILGFPAGICYTVGVGGHFSGG
GYGILMRKYGLGGDNVIDVRILLANGKIVDRKSMGGDLFW
ALRGGGAVSFGIVLAWKINLVDIPSTITIANVQMDYEQDS
TKRLVHQWQTIADKFDKDLLLFVRLQTGNSTTPGITKPSL
QASFAVVFLGGTDKLIPLVKKSFPDLGLARKDCVEMSWIQ
SILLENGFPTNSSLDVLLNRTQSVMFSFKAKADYVKEPIP
DDVVDKLAKSLYQEDLGTAVLQLFPYGGRMGEIPESETPF
PHRSGNLYELTYLARWVEKGNASETENHLKWTRSSYSYMA
PYVSKNPREAYLNYRDLDIGRNNYNGSTTYAQASIWGSKY
YKDNFKRLVYVKTMVDPSNFFRNEQSIPAYSL
Protein sequence translation from
nucleotide sequence
HL.Tea.v1.0.G019636.1 CBDAS homolog
in hops
SEQ ID NO: 24
MKLRDSSIFPSVIVEMIIISLSSTTKAYATLHDYQYTKTN
FIQCLSHHSSSNSSHNNDITKVVYSTTNSSYFSVLNFTII
NPRFSSPSTPKPLFIITPLHESHVQAAVVCSRKHGVQIRI
RSGGHDYEGLSYVSDVPFVVIDLINHRSITIDVEKRTAWV
QAGATLGELYYEINVKSKTLAFPAGACPTIGVGGHISGGG
YGSIFRKYGLAADNVIDAQIVDVEGRVLDRETMGEDLFWA
IRGGGGASFGVILAWKVRLVPVPETVTVFAINRNLEHNVT
KLVHRWQYIADKLHKDLLLAVRFQTVKVNSTQEGSYKKEL
QATFISVFLGRVDGLLDLMGKRFPELGLAREDCTEMSWIE
SALFVAGLPREQSPEILLDRTPQSRLSFKAKSDYVKEPIP
EKGLEGIWERLYEEEIGRGVVIMSPYGGKMSEISESELPF
GHRAGNLYKIQYLIYWEEEGNATVMEKHISWIRRLYYYMT
QFVSKNPRSAYINYRDLDIGTNSNNGTASYAQASIWGVKY
FGHNNFNRLVHVKTIVDPTNFFRNEQSIPPLRIEYSS
Protein sequence translation from
nucleotide sequence
HL.Tea.v1.0.G037793.1 CBDAS homolog
in hops
SEQ ID NO: 25
MKHSVFSYWFLCKIVNISLLSFSIRSTRADPHADFLQCFS
QYISNSTTIAKLIYTPNDPLYISILNSTIQNNRFSSPSTP
KPLIIITPLNSFHVQASILCSRKYGLQIRTRSGGHDFEGV
SYVSEVPFVIVDMRNLRSITIDVDNKTAWVDVGATLGELY
YRIAEKNENLSFPAGYCHTVGVGGHFSGGGYGALMRKYGL
AADNVIDAHLVNVDGEVLDRQSMGEDLFWAIRGGGGASFG
IILAWKIRLVPVPSKVTIVSINKNLEINETVKLYNKWQNI
AHKFDKDLLIFVRFTTMNSTDGQGKNKTAILTSFYSIFFG
GMDGLLALMEKSFPELDVKRKDCFEASWIEMIFYFNGFSS
GDKLEVLLGRTNEEKGFFKAKLDYVRKPIPETVIVKLLEK
LYNEDVGLGLIQMYPYGGKMDEIPESAIPFPHRVGFIYKI
LYLSQWEKEEEGERHLNWVRSVYNYMTPFVSKSPRASYLN
YRDFDLGTNNKNGPTSYGQASIWGKKYFDKNFKRLVHVKT
KVDPTNFFRNEQSIPPLSVRGL
Prepro alpha-CBCAS Protein Sequence
(The prepro sequence is underlined.
The start of the mature polypeptide
sequence is shown in bold)
SEQ ID NO: 26
MRFPSIFTTVLFAASSALAAPVNTTTEDETAQIPAEAVIG
YLDLEGDEDVAVLPFSNSTNNGLLFINTTIASIAAKEEGV
SLDKRANPQENFLKCFSEYIPNNPANPKFIYTQHDQLYMS
VLNSTIQNLRFTSDTTPKPLVIVTPSNVSHIQASILCSKK
VGLQIRTRSGGHDAEGLSYISQVPFAIVDLRNMHTVKVDI
HSQTAWVEAGATLGEVYYWINEMNENFSFPGGYCPTVGVG
GHFSGGGYGALMRNYGLAADNIIDAHLVNVDGKVLDRKSM
GEDLFWAIRGGGGENFGIIAAWKIKLVVVPSKATIFSVKK
NMEIHGLVKLFNKWQNIAYKYDKDLMLTTHFRTRNITDNH
GKNKTTVHGYFSSIFLGGVDSLVDLMNKSFPELGIKKTDC
KELSWIDTTIFYSGVVNYNTANFKKEILLDRSAGKKTAFS
IKLDYVKKLIPETAMVKILEKLYEEEVGVGMYVLYPYGGI
MDEISESAIPFPHRAGIMYELWYTATWEKQEDNEKHINWV
RSVYNFTTPYVSQNPRLAYLNYRDLDLGKTNPESPNNYTQ
ARIWGEKYFGKNFNRLVKVKTKADPNNFFRNEQSIPPLPP
RHH
Prepro alpha-CBCAS-HDEL (The prepro
sequence and HDEL sequences
are underlined.)
SEQ ID NO: 27
MRFPSIFTTVLFAASSALAAPVNTTTEDETAQIPAEAVIG
YLDLEGDEDVAVLPFSNSTNNGLLFINTTIASIAAKEEGV
SLDKRANPQENFLKCFSEYIPNNPANPKFIYTQHDQLYMS
VLNSTIQNLRFTSDTTPKPLVIVTPSNVSHIQASILCSKK
VGLQIRTRSGGHDAEGLSYISQVPFAIVDLRNMHTVKVDI
HSQTAWVEAGATLGEVYYWINEMNENFSFPGGYCPTVGVG
GHFSGGGYGALMRNYGLAADNIIDAHLVNVDGKVLDRKSM
GEDLFWAIRGGGGENFGIIAAWKIKLVVVPSKATIFSVKK
NMEIHGLVKLFNKWQNIAYKYDKDLMLTTHFRTRNITDNH
GKNKTTVHGYFSSIFLGGVDSLVDLMNKSFPELGIKKTDC
KELSWIDTTIFYSGVVNYNTANFKKEILLDRSAGKKTAFS
IKLDYVKKLIPETAMVKILEKLYEEEVGVGMYVLYPYGGI
MDEISESAIPFPHRAGIMYELWYTATWEKQEDNEKHINWV
RSVYNFTTPYVSQNPRLAYLNYRDLDLGKTNPESPNNYTQ
ARIWGEKYFGKNFNRLVKVKTKADPNNFFRNEQSIPPLPP
RHHHDEL
Pdi1-CBCAS (The Saccharomyces cerevisiae
Pdil signal sequence is
underlined)
SEQ ID NO: 28
MKFSAGAVLSWSSLLLASSVFAQQANPQENFLKCFSEYIP
NNPANPKFIYTQHDQLYMSVLNSTIQNLRFTSDTTPKPLV
IVTPSNVSHIQASILCSKKVGLQIRTRSGGHDAEGLSYIS
QVPFAIVDLRNMHTVKVDIHSQTAWVEAGATLGEVYYWIN
EMNENFSFPGGYCPTVGVGGHFSGGGYGALMRNYGLAADN
IIDAHLVNVDGKVLDRKSMGEDLFWAIRGGGGENFGIIAA
WKIKLVVVPSKATIFSVKKNMEIHGLVKLFNKWQNIAYKY
DKDLMLTTHFRTRNITDNHGKNKTTVHGYFSSIFLGGVDS
LVDLMNKSFPELGIKKTDCKELSWIDTTIFYSGVVNYNTA
NFKKEILLDRSAGKKTAFSIKLDYVKKLIPETAMVKILEK
LYEEEVGVGMYVLYPYGGIMDEISESAIPFPHRAGIMYEL
WYTATWEKQEDNEKHINWVRSVYNFTTPYVSQNPRLAYLN
YRDLDLGKTNPESPNNYTQARIWGEKYFGKNFNRLVKVKT
KADPNNFFRNEQSIPPLPPRHH
EasE-CBCAS (The berberine bridge-
associated easE signal sequence from
Aspergillus japonica is underlined.)
SEQ ID NO: 29
MGQSRGILGGVRQLILVILVGAYLSRLSAVDANPQENFLK
CFSEYIPNNPANPKFIYTQHDQLYMSVLNSTIQNLRFTSD
TTPKPLVIVTPSNVSHIQASILCSKKVGLQIRTRSGGHDA
EGLSYISQVPFAIVDLRNMHTVKVDIHSQTAWVEAGATLG
EVYYWINEMNENFSFPGGYCPTVGVGGHFSGGGYGALMRN
YGLAADNIIDAHLVNVDGKVLDRKSMGEDLFWAIRGGGGE
NFGIIAAWKIKLVVVPSKATIFSVKKNMEIHGLVKLFNKW
QNIAYKYDKDLMLTTHFRTRNITDNHGKNKTTVHGYFSSI
FLGGVDSLVDLMNKSFPELGIKKTDCKELSWIDTTIFYSG
VVNYNTANFKKEILLDRSAGKKTAFSIKLDYVKKLIPETA
MVKILEKLYEEEVGVGMYVLYPYGGIMDEISESAIPFPHR
AGIMYELWYTATWEKQEDNEKHINWVRSVYNFTTPYVSQN
PRLAYLNYRDLDLGKTNPESPNNYTQARIWGEKYFGKNFN
RLVKVKTKADPNNFFRNEQSIPPLPPRHH
Prepro alpha-CBCAS (Amino acids 87 to 545
of SEQ ID NO: 32. The prepro
sequence is underlined.)
SEQ ID NO: 30
MRFPSIFTTVLFAASSALAAPVNTTTEDETAQIPAEAVIG
YLDLEGDEDVAVLPFSNSTNNGLLFINTTIASIAAKEEGV
SLDKRPSNVSHIQASILCSKKVGLQIRTRSGGHDAEGLSY
ISQVPFAIVDLRNMHTVKVDIHSQTAWVEAGATLGEVYYW
INEMNENFSFPGGYCPTVGVGGHFSGGGYGALMRNYGLAA
DNIIDAHLVNVDGKVLDRKSMGEDLFWAIRGGGGENFGII
AAWKIKLVVVPSKATIFSVKKNMEIHGLVKLFNKWQNIAY
KYDKDLMLTTHFRTRNITDNHGKNKTTVHGYFSSIFLGGV
DSLVDLMNKSFPELGIKKTDCKELSWIDTTIFYSGVVNYN
TANFKKEILLDRSAGKKTAFSIKLDYVKKLIPETAMVKIL
EKLYEEEVGVGMYVLYPYGGIMDEISESAIPFPHRAGIMY
ELWYTATWEKQEDNEKHINWVRSVYNFTTPYVSQNPRLAY
LNYRDLDLGKTNPESPNNYTQARIWGEKYFGKNFNRLVKV
KTKADPNNFFRNEQSIPPLPPRHH*
CBCAS (amino acids 87-545 of SEQ ID NO: 32,
with methionine initiation codon)
SEQ ID NO: 31
MPSNVSHIQASILCSKKVGLQIRTRSGGHDAEGLSYISQV
PFAIVDLRNMHTVKVDIHSQTAWVEAGATLGEVYYWINEM
NENFSFPGGYCPTVGVGGHFSGGGYGALMRNYGLAADNII
DAHLVNVDGKVLDRKSMGEDLFWAIRGGGGENFGIIAAWK
IKLVVVPSKATIFSVKKNMEIHGLVKLFNKWQNIAYKYDK
DLMLTTHFRTRNITDNHGKNKTTVHGYFSSIFLGGVDSLV
DLMNKSFPELGIKKTDCKELSWIDTTIFYSGVVNYNTANF
KKEILLDRSAGKKTAFSIKLDYVKKLIPETAMVKILEKLY
EEEVGVGMYVLYPYGGIMDEISESAIPFPHRAGIMYELWY
TATWEKQEDNEKHINWVRSVYNFTTPYVSQNPRLAYLNYR
DLDLGKTNPESPNNYTQARIWGEKYFGKNFNRLVKVKTKA
DPNNFFRNEQSIPPLPPRHH*
Full length CBCAS synthase
SEQ ID NO: 32
MNCSTFSFWFVCKIIFFFLSFNIQISIANPQENFLKCFSE
YIPNNPANPKFIYTQHDQLYMSVLNSTIQNLRFTSDTTPK
PLVIVTPSNVSHIQASILCSKKVGLQIRTRSGGHDAEGLS
YISQVPFAIVDLRNMHTVKVDIHSQTAWVEAGATLGEVYY
WINEMNENFSFPGGYCPTVGVGGHFSGGGYGALMRNYGLA
ADNIIDAHLVNVDGKVLDRKSMGEDLFWAIRGGGGENFGI
IAAWKIKLVVVPSKATIFSVKKNMEIHGLVKLFNKWQNIA
YKYDKDLMLTTHFRTRNITDNHGKNKTTVHGYFSSIFLGG
VDSLVDLMNKSFPELGIKKTDCKELSWIDTTIFYSGVVNY
NTANFKKEILLDRSAGKKTAFSIKLDYVKKLIPETAMVKI
LEKLYEEEVGVGMYVLYPYGGIMDEISESAIPFPHRAGIM
YELWYTATWEKQEDNEKHINWVRSVYNFTTPYVSQNPRLA
YLNYRDLDLGKTNPESPNNYTQARIWGEKYFGKNFNRLVK
VKTKADPNNFFRNEQSIPPLPPRHH
hSOD-TKS (hSOD sequence is underlined)
SEQ ID NO: 33
MATKAVCVLKGDGPVQGIINFEQKESNGPVKVWGSIKGLT
EGLHGFHVHEFGDNTAGCTSAGPHENPLSRKHGGPKDEER
HVGDLGNVTADKDGVADVSIEDSVISLSGDHCIIGRTLVV
HEKADDLGKGGNEESTKTGNAGSRLACGVIGIAQPRMNHL
RAEGPASVLAIGTANPENILLQDEFPDYYFRVTKSEHMTQ
LKEKFRKICDKSMIRKRNCFLNEEHLKQNPRLVEHEMQTL
DARQDMLVVEVPKLGKDACAKAIKEWGQPKSKITHLIFTS
ASTTDMPGADYHCAKLLGLSPSVKRVMMYQLGCYGGGTVL
RIAKDIAENNKGARVLAVCCDIMACLFRGPSESDLELLVG
QAIFGDGAAAVIVGAEPDESVGERPIFELVSTGQTILPNS
EGTIGGHIREAGLIFDLHKDVPMLISNNIEKCLIEAFTPI
GISDWNSIFWITHPGGKAILDKVEEKLHLKSDKFVDSRHV
LSEHGNMSSSTVLFVMDELRKRSLEEGKSTTGDGFEWGVL
FGFGPGLTVERVVVRSVPIKYAS

What is claimed is:

1. A modified recombinant host cell comprising a first exogenous polynucleotide that encodes a CoA-transferase that converts an aliphatic carboxylic acid to an acyl CoA thioester, wherein the modified recombinant host cell is engineered to produce a cannabinoid product.

2. The modified recombinant host cell of claim 1, wherein the CoA-transferase is selected from the group consisting of R. hominis butyryl-CoA:acetate CoA-transferase, E. coli acetyl-CoA:acetoacetyl CoA-transferase, and C. necator H16 propionate CoA-transferase.

3. A modified recombinant host cell comprising a first exogenous polynucleotide that encodes a CoA-ligase,
wherein the CoA-ligase is selected from the group consisting of M. avium mig medium chain acyl-CoA-ligase, and A. thaliana AT4g05160 coumarate acyl-CoA-ligase, and wherein the modified recombinant host cell is engineered to produce a cannabinoid product.

4. The modified recombinant host cell of claim 2, wherein the modified recombinant host cell further comprises one or more exogenous polynucleotides selected from the group consisting of:
a second exogenous polynucleotide that encodes a polyketide synthase (PKS), and
a third exogenous polynucleotide that encodes a 2-alkyl-4,6-dihydroxybenzoic acid cyclase.

5. The modified recombinant host cell of claim 4, wherein the PKS is a type I PKS, a type II PKS, or a type III PKS.

6. The modified recombinant host cell of claim 4, wherein the 2-alkyl-4,6-dihydroxybenzoic acid cyclase is olivetolic acid cyclase, an AtHS1 polypeptide, or the N-terminal domain of a BenH polypeptide.

7. The modified recombinant host cell of claim 4, wherein the modified recombinant host cell further comprises a fourth polynucleotide that encodes a prenyltransferase.

8. The modified recombinant host cell of claim 7, wherein the prenyltransferase is geranylpyrophosphate:olivetolate geranyltransferase.

9. The modified recombinant host cell of claim 7, wherein multiple copies of the fourth polynucleotide are integrated into the host cell genome.

10. The modified recombinant host cell of claim 1, wherein expression of one or more of the exogenous polynucleotides is driven by an ADH2 promoter, an ADH1 promoter, a GAL1 promoter, a MET25 promoter, a CUP1 promoter, a GPD promoter, a PGK promoter, a PYK promoter, a TPI promoter, a TEF1 promoter, or an FBA1 promoter.

11. The modified recombinant host cell of claim 1, wherein at least two of the exogenous polynucleotides are present in the same autonomously replicating expression vector and expressed as a multicistronic mRNA.

12. The modified recombinant host cell of claim 1, wherein the host cell is genetically modified to overexpress one or more mevalonate pathway enzymes.

13. The modified recombinant host cell of claim 12, wherein the mevalonate pathway enzymes are selected from the group consisting of erg10, erg13, thmgr, erg12, erg8, mvd1, idi1, erg20 F96WN127W, E. faecalis mvaE, and E. faecalis mvaS.

14. The modified recombinant host cell of claim 1, wherein the modified recombinant host cell is a cell selected from the group consisting of Saccharomyces cerevisiae, Kluyveromyces lactis, Kluyveromyces marxianus, Pichia pastoris, Yarrowia hpolytica, Hansenula polymorpha and Aspergillus

15. A method of producing a cannabinoid product, the method comprising:
culturing a modified recombinant host cell comprising a first exogenous polynucleotide that encodes a CoA-transferase that converts an aliphatic carboxylic acid to an acyl CoA thioester under conditions in which the CoA-transferase encoded by the exogenous polynucleotide is expressed and the acyl CoA thioester is produced; and
converting the acyl CoA thioester to the cannabinoid product.

16. The method of claim 15, wherein the CoA-transferase is selected from the group consisting of R. hominis butyryl-CoA:acetate CoA-transferase, E. coli acetyl-CoA:acetoacetyl-CoA-transferase, and C. necator H16 propionate CoA-transferase.

17. A method of producing a cannabinoid product, the method comprising:
culturing a modified recombinant host cell comprising a first exogenous polynucleotide that encodes a CoA-ligase that converts an aliphatic carboxylic acid to an acyl CoA thioester under conditions in which the CoA-ligase encoded by the exogenous polynucleotide is expressed and the acyl CoA thioester is produced; and
converting the acyl CoA thioester to the cannabinoid product;
wherein the CoA-ligase is selected from the group consisting of M. avium mig medium chain acyl-CoA-ligase and A. thaliana AT4g05160 coumarate acyl-CoA-ligase.

18. The method of claim 15, wherein the aliphatic carboxylic acid is a C₂₋₅ carboxylic acid.

19. The method of claim 15, wherein the aliphatic carboxylic acid is a C₆₋₂₀ carboxylic acid.

20. The method of claim 15, wherein the aliphatic carboxylic acid comprises a carbon-carbon double bond, a hydroxy group, a halogen, deuterium, tritium, or a combination thereof.

21. The method of claim 16, wherein the modified recombinant host cell further comprises one or more exogenous polynucleotides selected from the group consisting of:
a second exogenous polynucleotide that encodes a polyketide synthase (PKS) that produces a polyketide from the acyl CoA thioester and malonyl CoA, and
a third exogenous polynucleotide that encodes a 2-alkyl-4,6-dihydroxybenzoic acid cyclase;
wherein culturing the modified recombinant host cell comprises expressing products encoded by the second and third exogenous polynucleotides and converting the acyl CoA thioester to a 2-alkyl-4,6-dihydroxybenzoic acid or a 5-alkyl-benzene-1,3-diol; and
wherein converting the acyl CoA thioester to the cannabinoid product comprises converting the 2-alkyl-4,6-dihydroxybenzoic acid or the 5-alkyl-benzene-1,3-diol to the cannabinoid product.

22. The method of claim 21, wherein the 2-alkyl-4,6-dihydroxybenzoic acid is divarinic acid.

23. The method of claim 21, wherein the PKS is a type I PKS, a type II PKS, ora type III PKS.

24. The method of claim 21, wherein the 2-alkyl-4,6-dihydroxybenzoic acid cyclase is olivetolic acid cyclase, an AtHS1 polypeptide, or the N-terminal domain of a BenH polypeptide.

25. The method of claim 21, wherein converting the 2-alkyl-4,6-dihydroxybenzoic acid or the 5-alkyl-benzene-1,3-diol to the cannabinoid product comprises producing a prenylated 2-alkyl-4,6-dihydroxybenzoic acid or a prenylated 5-alkyl-benzene-1,3-diol.

26. The method of claim 25, wherein the modified recombinant host cell further comprises fourth polynucleotide that encodes a prenyltransferase, and wherein culturing the modified recombinant host cell comprises expressing the prenyltransferase encoded by the fourth exogenous polynucleotides and producing the prenylated 2-alkyl-4,6-dihydroxybenzoic acid or the prenylated 5-alkyl-benzene-1,3-diol.

27. The method of claim 26, wherein the prenyltransferase is geranylpyrophosphate:olivetolate geranyltransferase.

28. The method of claim 26, wherein multiple copies of the fourth polynucleotide are integrated into the host cell genome.

29. The method of claim 25, wherein producing the prenylated 2-alkyl-4,6-dihydroxybenzoic acid or the prenylated 5-alkyl-benzene-1,3-diol comprises:
forming a reaction mixture comprising 1) the 2-alkyl-4,6-dihydroxybenzoic acid or the 5-alkyl-benzene-1,3-diol and 2) geraniol, an activated geraniol, or citral, and
maintaining the reaction mixture under conditions sufficient to form the prenylated 2-alkyl-4,6-dihydroxybenzoic acid or the prenylated 5-alkyl-benzene-1,3-diol.

30. The method of claim 29, wherein the reaction mixture further comprises a diamine.

31. The method of claim 15, wherein expression of one or more of the exogenous polynucleotides is driven by an ADH2 promoter, an ADH1 promoter, a GAL1 promoter, a MET25 promoter, a CUP1 promoter, a GPD promoter, a PGK promoter, a PYK promoter, a TPI promoter, a TEF1 promoter, or an FBA1 promoter.

32. The method of claim 15, wherein at least two of the exogenous polynucleotides are present in the same autonomously replicating expression vector and expressed as a multicistronic mRNA.

33. The method of claim 15, wherein the host cell is genetically modified to overexpress one or more mevalonate pathway enzymes.

34. The method of claim 33, wherein the mevalonate pathway enzymes are selected from the group consisting of erg10, erg13, thmgr, erg12, erg8, mvd1, idi1, erg20 F96WN127W, E. faecalis mvaE, and E. faecalis mvaS.

35. The method claim 15, wherein the modified recombinant host cell is a cell selected from the group consisting of Saccharomyces cerevisiae, Kluyveromyces lactis, Kluyveromyces marxianus, Pichia pastoris, Yarrowia lipolytica, Hansenula polymorpha and Aspergillus.


SEQUENCE LISTING (36 sequences)

SEQ ID NO: 1 - protein, 446 aa, Roseburia hominis
MDFREEYKQKLVSADEAVKLIKSGDWVDYGWCTNTVDALDQALAKRTDELTDVKLRGGILMKPLAVFAREDAGEHFCWNSWHMSGIERKMINRGVAYYCPIRYSELPRYYRELDCPDDVAMFQVAPMDAHGYFNFGPSASHLGAMCERAKHIIVEVNENMPRCLGGTECGIHISDVTYIVEGSNPPIGELGAGGPATDVDKAVAKLIVDEIPNGACLQLGIGGMPNAVGSLIAESDLKDLGVHTEMYVDAFVDIAKAGKINGSKKNIDRYRQTYAFGAGTKKMYDYLDDNPELMSAPVDYTNDIRSISALDNFISINNAVDIDLYGQVNAESAGIKQISGAGGQLDFVLGAYLSKGGKSFICLSSTFKTKDGQVQSRIRPTLANGSIVTDARPNTHYVVTEYGKVNLKGLSTWQRAEALISIAHPDFRDDLIKEAEQMHIWRRSNR

SEQ ID NO: 2 - protein, 216 aa, Escherichia coli
MDAKQRIARRVAQELRDGDIVNLGIGLPTMVANYLPEGIHITLQSENGFLGLGPVTTAHPDLVNAGGQPCGVLPGAAMFDSAMSFALIRGGHIDACVLGGLQVDEEANLANWVVPGKMVPGMGGAMDLVTGSRKVIIAMEHCAKDGSAKILRRCTMPLTAQHAVHMLVTELAVFRFIDGKMWLTEIADGCDLATVRAKTEARFEVAADLNTQRGDL

SEQ ID NO: 3 - protein, 180 aa, Escherichia coli
MKTKLMTLQDATGFFRDGMTIMVGGFMGIGTPSRLVEALLESGVRDLTLIANDTAFVDTGIGPLIVNGRVRKVIASHIGTNPETGRRMISGEMDVVLVPQGTLIEQIRCGGAGLGGFLTPTGVGTVVEEGKQTLTLDGKTWLLERPLRADLALIRAHRCDTLGNLTYQLSARNFNPLIAL

SEQ ID NO: 4 - protein, 542 aa, Cupriavidus necator
MKVITAREAAALVQDGWTVASAGFVGAGHAEAVTEALEQRFLQSGLPRDLTLVYSAGQGDRGARGVNHFGNAGMTASIVGGHWRSATRLATLAMAEQCEGYNLPQGVLTHLYRAIAGGKPGVMTKIGLHTFVDPRTAQDARYHGGAVNERARQAIAEGKACWVDAVDFRGDEYLFYPSFPIHCALIRCTAADARGNLSTHREAFHHELLAMAQAAHNSGGIVIAQVESLVDHHEILQAIHVPGILVDYVVVCDNPANHQMTFAESYNPAYVTPWQGEAAVAEAEAAPVAAGPLDARTIVQRRAVMELARRAPRVVNLGVGMPAAVGMLAHQAGLDGFTLTVEAGPIGGTPADGLSFGASAYPEAVVDQPAQFDFYEGGGIDLAILGLAELDGHGNVNVSKFGEGEGASIAGVGGFINITQSARAVVFMGTLTAGGLEVRAGDGGLQIVREGRVKKIVPEVSHLSFNGPYVASLGIPVLYITERAVFEMRAGADGEARLTLVEIAPGVDLQRDVLDQCSTPIAVAQDLREMDARLFQAGPLHL

SEQ ID NO: 5 - protein, 550 aa, Mycobacterium avium
MSDTTTAFTVPAVAKAVAAAIPDRELIIQGDRRYSYRQVIERSNRLAAYLHSQGLGCHTEREALAGHEVGQDLLGLYAYNGNEFVEALLGAFAARVAPFNVNFRYVKSELHYLLADSEATALIYHAAFAPRVAEILPDLPRLRVLIQIADESGNELLDGAVDYEDALASVSAEPPPVRHCPDDLYVLYTGGTTGMPKGVLWRQHDIFMTSFGGRNLMTGEPSSSIDEIVQRAASGPGTKLMILPPLIHGAAQWSVMTAITTGQTVVFPTVVDHLDAEDVVRTIEREKVMVVTVVGDAMARPLVAAIEKGIADVSSLAVVANGGALLTPFVKQRLIEVLPNAVVVDGVGSSETGAQMHHMSTPGAVATGTFNAGPDTFVAAEDLSAILPPGHEGMGWLAQRGYVPLGYKGDAAKTAKTFPVIDGVRYAVPGDRARHHADGHIELLGRDSVCINSGGEKIFVEEVETAIASHPAVADVVVAGRPSERWGQEVVAVVALSDGAAVDAGELIAHASNSLARYKLPKAIVFRPVIERSPSGKADYRWAREQAVDG

SEQ ID NO: 6 - protein, 544 aa, Arabidopsis thaliana
MEKSGYGRDGIYRSLRPTLVLPKDPNTSLVSFLFRNSSSYPSKLAIADSDTGDSLTFSQLKSAVARLAHGFHRLGIRKNDVVLIFAPNSYQFPLCFLAVTAIGGVFTTANPLYTVNEVSKQIKDSNPKIIISVNQLFDKIKGFDLPVVLLGSKDTVEIPPGSNSKILSFDNVMELSEPVSEYPFVEIKQSDTAALLYSSGTTGTSKGVELTHGNFIAASLMVTMDQDLMGEYHGVFLCFLPMFHVFGLAVITYSQLQRGNALVSMARFELELVLKNIEKFRVTHLWVVPPVFLALSKQSIVKKFDLSSLKYIGSGAAPLGKDLMEECGRNIPNVLLMQGYGMTETCGIVSVEDPRLGKRNSGSAGMLAPGVEAQIVSVETGKSQPPNQQGEIWVRGPNMMKGYLNNPQATKETIDKKSWVHTGDLGYFNEDGNLYVVDRIKELIKYKGFQVAPAELEGLLVSHPDILDAVVIPFPDEEAGEVPIAFVVRSPNSSITEQDIQKFIAKQVAPYKRLRRVSFISLVPKSAAGKILRRELVQQVRSKM

SEQ ID NO: 7 - protein, 744 aa, Saccharomyces cerevisiae
MAAPDYALTDLIESDPRFESLKTRLAGYTKGSDEYIEELYSQLPLTSYPRYKTFLKKQAVAISNPDNEAGFSSIYRSSLSSENLVSCVDKNLRTAYDHFMFSARRWPQRDCLGSRPIDKATGTWEETFRFESYSTVSKRCHNIGSGILSLVNTKRKRPLEANDFVVAILSHNNPEWILTDLACQAYSLTNTALYETLGPNTSEYILNLTEAPILIFAKSNMYHVLKMVPDMKFVNTLVCMDELTHDELRMLNESLLPVKCNSLNEKITFFSLEQVEQVGCFNKIPAIPPTPDSLYTISFTSGTTGLPKGVEMSHRNIASGIAFAFSTFRIPPDKRNQQLYDMCFLPLAHIFERMVIAYDLAIGFGIGFLHKPDPTVLVEDLKILKPYAVALVPRILTRFEAGIKNALDKSTVQRNVANTILDSKSARFTARGGPDKSIMNFLVYHRVLIDKIRDSLGLSNNSFIITGSAPISKDTLLFLRSALDIGIRQGYGLTETFAGVCLSEPFEKDVGSCGAIGISAECRLKSVPEMGYHADKDLKGELQIRGPQVFERYFKNPNETSKAVDQDGWFSTGDVAFIDGKGRISVIDRVKNFFKLAHGEYIAPEKIENIYLSSCPYITQIFVFGDPLKTFLVGIVGVDVDAAQPILAAKHPEVKTWTKEVLVENLNRNKKLRKEFLNKINKCTDGLQGFEKLHNIKVGLEPLTLEDDVVTPTFKIKRAKASKFFKDTLDQLYAEGSLVKTEKL

SEQ ID NO: 8 - protein, 566 aa, Escherichia coli
MHPTGPHLGPDVLFRESNMKVTLTFNEQRRAAYRQQGLWGDASLADYWQQTARAMPDKIAVVDNHGASYTYSALDHAASCLANWMLAKGIESGDRIAFQLPGWCEFTVIYLACLKIGAVSVPLLPSWREAELVWVLNKCQAKMFFAPTLFKQTRPVDLILPLQNQLPQLQQIVGVDKLAPATSSLSLSQIIADNTSLTTAITTHGDELAAVLFTSGTEGLPKGVMLTHNNILASERAYCARLNLTWQDVFMMPAPLGHATGFLHGVTAPFLIGARSVLLDIFTPDACLALLEQQRCTCMLGATPFVYDLLNVLEKQPADLSALRFFLCGGTTIPKKVARECQQRGIKLLSVYGSTESSPHAVVNLDDPLSRFMHTDGYAAAGVEIKVVDDARKTLPPGCEGEEASRGPNVFMGYFDEPELTARALDEEGWYYSGDLCRMDEAGYIKITGRKKDIIVRGGENISSREVEDILLQHPKIHDACVVAMSDERLGERSCAYVVLKAPHHSLSLEEVVAFFSRKRVAKYKYPEHIVVIEKLPRTTSGKIQKFLLRKDIMRRLTQDVCEEIE

SEQ ID NO: 9 - protein, 2700 aa, Artificial Sequence
  Description of Artificial Sequence Synthetic polypeptide
MTTHALTERATLVDWIEHHARARPLAEALFFCGHGADDLRLGYGALSERVRRCAAALQQRGAAGSTALILFPSGIDYVVALLACFYAGVTGVPVNLPGVSRVRRVLPKLGDITRDCRPAVVLTHTAIERASGNDLRDFAAGHGLDILHLDTLGGEAAAWVRPALTPESIAFLQYTSGSTGSPKGVVNRHGALLRNLQFLGRLTRPQDRAPEDTAVASWLPLFHDLGLIMGILLPLAYGNRAVYMAPMAFVADPLRWLEIATAERATALPCPSFALRLCADEARRAAPARTAGIDLSSVQCLMPAAEPVLPSQIEAFQAAFAAHGMRREAIRPAYGLAEATLLVSANVDDAPPHRIDVETAPLEQGRAVVHPAAAPMPAAGRRRYVSNGREFDGQDVRIVDPRTCATLPEGTVGEIWISGPCIAGGYWNKAELNREIFMAETPGAGDRRYLRTGDMGFLHGGHLFVTGRLKDMMLFRGQCHYPNDIEATSGRAHAAAIPESGAAFSIQAEDEAGERLVIVQEVRKQAGIDPRDIATAVRAAVAEGHALGVHAVVLIRKGTLPRTTSGKVRRAAVREAWLAGTLQTLWQDDIDNLAVPPTPAQETAAAPADAALLAALAPLDAARRQQHLVQWLAARAAAALGTVAARAIRPEASLFGYGLDSMSATRLAAVAAAASGLALPDSLLFDHPSLDGLAGWLLQAMEQARHLPPAPGGRDRAMPAPRPAAHRHGDGQDPIAIIGMAFRLPGENGHDADTDAAFWRLLDGAGCAIRPMPAERFRAPAGMPGFGAYLNQVDRFDAAFFGMSPREAMNTDPQQRLLLEVAWHALEDAGLPPGDLRGSDSGVFVGIGTADYGHLPFISGDDAHFDAYWGTGTSFAAACGRLSFTFGWEGPSMAVDTACSASHSALHLAVQALRARECGMALSAGVKLQLLPEIDRVLHKAGMLAADGRCKTLDASADGYVRGEGCVVLVLKRLSDALADGDAIRAVIRDTLVRQDGAGSSLSAPNGEAQQRLLSLALARAGLAPSEIDYIELHGTGTRLGDPIEYQSVADVFGGRAPDDPLWIGSVKTNIGHLESAAGAAGLVKTVLALEQARIPPLVGLKGINPLIDLDAIPARAPAHTVDWPARQAVRRAGVTSYGFAGTIAHVILEQAPQAPVAQAAGTEPTRGPHLFLLSARSPDALRRLAAAYRDTLAGTADLAVLANGMARQREHHALRAAVVASDHDECARALDRLAAPDAAAPEAVTRAPRVGFLFTGQGSQYAGMTRALYAAQPDFRAALDAADAALAPHLGRSILALMHDDAQRDALQQTAHAQPALFACGYALAAMWQAWGVVPAVLVGHSIGEFAAMVVAGAMTLEDAARLIVRRGALMQALPAGGAMLAARATPRHAHDLLAALAPAVAAEVSLAAINGPQDVVFSGSAAGIDAVRARLDAQQLDARPLAVSHAFHSPLLDPMLGDWAEACADAQSAPPRIPLISTLTGAPMTTAPDAAYWSAHARQPVRFAEALARAGADCDVLLEIGAHAVLSALAQRNQLAQPWPHPVACVASLLRGTDDSRAVAQACAELYLRGQPFDWDRLFAGPLPSPRALPRYPFDRQSHWLEYDEDAPRTPLPMQPQPERAAPRPVERYAVQWEPFAPSAGDGHASTYWIVAADAADAGPADAGRLAARLSGPARDVHVLSPSQWADAADRIADDDVVIYLAGWPARASDAAAVAGSRHVWQLTECVRTLQRLRKTPRILLPTLHGQSPDGAPCDPLQAALWGAARPLSLEYPGPAWLLADCAGESPLETLADALPALLPLFGKEEAVALRAGGWLRPRLTPQAAPERAPCVTLRADGLYLVAGAYGALGRHTTDWLAAHGATHLVLAGRRAPPAGWQARLALLRAQGVRIDPVDADLAEAADVERLFDAVAALEATTGRTLAGVFHCAGTSRFNDLAGLTTDDCAAVTGAKMTGAWLLHEQTRARRLDWFVCFTSISGVWGSRLQIPYGAANAFQDALVRLRRAQGLPALAVAWGPWGGGAGMSEVDDALLQLLRAAGIRRLAPSRYLATLDHLLGHAEHADGLPADGTCVVAEVDWQQFIPLFALYNPIGTFERCRTDTATHATAAPSALIALDSGARADAVRAFVIAELARTLRVAPSQLTPDIELLKLGMDSILVMDFSRRCESGLGVKCELKAIFERNTPGGLASYLLERLEHAPQGAVPAPAAAEPIVHAPDHAHLPFPLTELQHAYWIGRQGHYALGGVACHAYLEADAADGLDLGLLERCWNALVARHGALRLVIDESGQQRILPRVPAYRIRVANLGAATPQALAAHCDDWRQAMSHQVLDAAQWPLFDVRATHLPGGATRLHIGIDMLINDATSGQIIWDELAALYRAGGDLERAGLAPFEISFRDYVLAKYVHSEARRAARESAKAYWLGQLETLPPAPQLPLRAEALHRAAPRFSRRQHRLSAPQWQSLRDRAAASGCTPASLLIAVFAEVLSAWSTEPRFTLNLTTFDRLPWHADVPRLLGDFTAVTLLPLDCAAPLPFGQRAAAVNGAVLEHLQHRAFSAVDVLREWNRGRERQDAVSMPVVFTSQLGMSDPTKGAARASVLGTVGYGISQTPQVWLDHQACELDGALIYNWDAVDALFQPGVLDAMFDAYNRMLERLAADADAWLEPLPALLPQAQREVRARVNASTAPLPERCLDQLFFDQA

SEQ ID NO: 10 - protein, 832 aa, Artificial Sequence
  Description of Artificial Sequence Synthetic polypeptide
MMTITTDRTPPAAGAALDRNRSAYAGLADVLERAGLAEHALYLNWGYRPVDGQPDWAARELPPGELGRMQARLVLEVLGDTPLDGRRVLDVGCGRGGALALMGRLHAPAALAGADISAANIAYCRKRHTHPRLRFQIADACRLPYPDSSMDVVFNLESSGAYPDIGAFFHHVHRILRVGGRFCLADVFDADSVAWVRAALEQAGFTLERERSIPAQVRAARERASPGIWRRLDTALTALDAPGLRRELERYLAAPSSGLFQALEDGRVDYRLFHWRKTCPAAGRIDADVIARLATRSARLDAALQDRAPSAAAPQSPAPGPANASASAWFPFTAPDAQAGFNVFALPYAGGGASVYRAWTLPRRPGAAPWQLCPVQLPGRESRFGEPLIDDMATLADRLADAIGPYAHRPWALLGCSLGCKIAFEVARRFARQGRPPALLFLMACPAPGLPLGRRISTRAEADFAREVCHLGGTPPEVLADAEMMRTLMPILRNDSALAEHYVAAEDATVNVPIVMVAAGDDHLVTVEEARRWQRHAGAGFDWRLVDGGHFFLRQRRRELTDWLLDALRRGERTLPVQTTTTDVPDVPCSTPEQPRDPSRMPAPGASANLVLAPGEILVVTAPRSLAARLTPAVLSDDEQRQLARFAFDADRERYLAAHWAKRRVLGALLAAAPRSLRFGAQAGGKPYLIGEALHFSLSHSGDRVAVAVCRHAPVGVDIEQARGIACHASAARIMHPLDRIAPQCETPEDRFLAAWSLKEAVAKCTGAGLALPFDSLRLAFAGNGRYGCLLGTHAAWEAHHQHEDGVHLAVASATPWAALRILPLDAALAEG

SEQ ID NO: 11 - protein, 394 aa, Streptomyces sp.
MAGRTATRRITLFDPERFRCRIAAECDFDAAALGLTPQEIRRMDRAVQMAVAATGEALADAGVGEGDLDPARTGVTIGNAVGSTMMMEEEYVVISDGGRKWLCDEEYGVRHLYGAVIPSTAGVEVARRVGAEGPTAVVSTGCTSGLDAVGHAAQLIEEGSADVVIGGATDAPISPITVACFDSLKATSTRNDDAEHACRPFDRDRDGLVLGEGSAVFVMEARERAVRRGAKIYCEVAGYAGRANAYHMTGLKPDGRELAEAIDRAMAQAGISAEDIDYVNAHGSGTRQNDRHETAAFKRSLRDHARRVPVSSIKSMVGHSLGAIGAIEVAASALAIEHGVVPPTANLTTPDPECDLDYVPREAREHPTDVVLSVGSGFGGFQSAVVLISPRSRR

SEQ ID NO: 12 - protein, 336 aa, Streptomyces sp.
MSQLSLSQAAPAGGSRIRGVGAYRPARVVTNEEIAPRIGVAPEWIARRSGIHTRRFAGPDEPLAMMAATASEKALAAAGLSADEVDCVLVATISHLLQMPALAVDVAHRLGAAPTAAFDLSAACAGFCHGVAIADSMVRSGTAHNVLLVGADRMTDVVDADDPATAFLFADGAGAVVIGPSETPGIGPVAWGSDGERMDAITMTGHWTPSLRTNPELPWPYLCMTGWKVFRWATETMGQAARDAIERAGVTSEELSAFIPHQANGLITDALAKDIGLTADTAIARDITDSGNTSGASIPMAMERLLASGQARSGEAALLIGFGSGLVHAGQVVLLP

SEQ ID NO: 13 - protein, 409 aa, Streptomyces sp.
MTVITGLGVVAPTGVGLDDYWATTLAGKSGIDRIRRFDPSGYTAQLAGQVDDFEATDHVPSKLLAQTDRMTHFAFAGANMALADAHVDLADFPEYERAVVTANSSGGVEYGQHELQKMWSGGPMRVSAYMSVAWFYAATTGQLSIHHGLRGPCGLIATEQAGGLDALGHARRLLRRGARIAVTGGTDAPLSPASMVAQLATGLLSSNPDPTAAYLPFDDRAAGYVPGEGGAIMIMEPAEHALRRGAERIYGEIAGYAATFDPAPGTGRGPTLGRAIRNALDDARIAPSEVDLVFADGSGTPAMDRAEAEALTEVFGPRGVPVTVPKAATGRMYSGGGALDVATALLAMRDGVAPPTPHVTELASDCPLDLVRTEPRELPIRHALVCARGVGGFNAALVLRRGDLTTPEH

SEQ ID NO: 14 - protein, 86 aa, Streptomyces sp.
MSTLSVEKLLEIMRATQGESADTSGLTEDVLDKPFTDLNVDSLAVLEVVTQIQDEFKLRIPDSAMEGMETPRQVLDYVNERLEEAA

SEQ ID NO: 15 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 16 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 17 - protein, 99 aa, Cannabis sativa
AVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPR

SEQ ID NO: 18 - protein, 95 aa, Artificial Sequence
  Description of Artificial Sequence Synthetic polypeptide
AVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFD

SEQ ID NO: 19 - protein, 109 aa, Arabidopsis thaliana
MEEAKGPVKHVLLASFKDGVSPEKIEELIKGYANLVNLIEPMKAFHWGKDVSIENLHQGYTHIFESTFESKEAVAEYIAHPAHVEFATIFLGSLDKVLVIDYKPTSVSL

SEQ ID NO: 20 - protein, 146 aa, Streptomyces sp.
AGRTDNSVVIDAPVQLVWDMTNDVSQWAVLFEEYAESEVLAVDGDTVRFRLTTQPDEDGKQWSWVSERTRDLENRTVTARRLDNGLFEYMNIRWEYTEGPDGVRMRWIQEFSMKPSAPVDDSGAEDHLNRQTVKEMARIKKLIEEA

SEQ ID NO: 21 - protein, 320 aa, Artificial Sequence
  Description of Artificial Sequence Synthetic polypeptide
MSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 22 - protein, 475 aa, Artificial Sequence
  Description of Artificial Sequence Synthetic polypeptide
MATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQPRSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 23 - protein, 472 aa, Artificial Sequence
  Description of Artificial Sequence Synthetic polypeptide
MNFRFSSPSLPKPSVIITPFHVSQIKATLMCSKKHGLQIRTRSGGHDSDGLSYISDVPYVVIDLRNLSKVKVDVHDKTAWVQAGATIGEVYYNIAKKSPILGFPAGICYTVGVGGHFSGGGYGILMRKYGLGGDNVIDVRILLANGKIVDRKSMGGDLFWALRGGGAVSFGIVLAWKINLVDIPSTITIANVQMDYEQDSTKRLVHQWQTIADKFDKDLLLFVRLQTGNSTTPGITKPSLQASFAVVFLGGTDKLIPLVKKSFPDLGLARKDCVEMSWIQSILLFNGFPTNSSLDVLLNRTQSVMFSFKAKADYVKEPIPDDVVDKLAKSLYQEDLGTAVLQLFPYGGRMGEIPESETPFPHRSGNLYELTYLARWVEKGNASETENHLKWTRSSYSYMAPYVSKNPREAYLNYRDLDIGRNNYNGSTTYAQASIWGSKYYKDNFKRLVYVKTMVDPSNFFRNEQSIPAYSL

SEQ ID NO: 24 - protein, 557 aa, Artificial Sequence
  Description of Artificial Sequence Synthetic polypeptide
MKLRDSSIFPSVIVFMIIISLSSTTKAYATLHDYQYTKTNFIQCLSHHSSSNSSHNNDITKVVYSTTNSSYFSVLNFTIINPRFSSPSTPKPLFIITPLHESHVQAAVVCSRKHGVQIRIRSGGHDYEGLSYVSDVPFVVIDLINHRSITIDVEKRTAWVQAGATLGELYYEINVKSKTLAFPAGACPTIGVGGHISGGGYGSIFRKYGLAADNVIDAQIVDVEGRVLDRETMGEDLFWAIRGGGGASFGVILAWKVRLVPVPETVTVFAINRNLEHNVTKLVHRWQYIADKLHKDLLLAVRFQTVKVNSTQEGSYKKELQATFISVFLGRVDGLLDLMGKRFPELGLAREDCTEMSWIESALFVAGLPREQSPEILLDRTPQSRLSFKAKSDYVKEPIPEKGLEGIWERLYEEEIGRGVVIMSPYGGKMSEISESELPFGHRAGNLYKIQYLIYWEEEGNATVMEKHISWIRRLYYYMTQFVSKNPRSAYINYRDLDIGTNSNNGTASYAQASIWGVKYFGHNNFNRLVHVKTIVDPTNFFRNEQSIPPLRIEYSS

SEQ ID NO: 25 - protein, 542 aa, Artificial Sequence
  Description of Artificial Sequence Synthetic polypeptide
MKHSVFSYWFLCKIVNISLLSFSIRSTRADPHADFLQCFSQYISNSTTIAKLIYTPNDPLYISILNSTIQNNRFSSPSTPKPLIIITPLNSFHVQASILCSRKYGLQIRTRSGGHDFEGVSYVSEVPFVIVDMRNLRSITIDVDNKTAWVDVGATLGELYYRIAEKNENLSFPAGYCHTVGVGGHFSGGGYGALMRKYGLAADNVIDAHLVNVDGEVLDRQSMGEDLFWAIRGGGGASFGIILAWKIRLVPVPSKVTIVSINKNLEINETVKLYNKWQNIAHKFDKDLLIFVRFTTMNSTDGQGKNKTAILTSFYSIFFGGMDGLLALMEKSFPELDVKRKDCFEASWIEMIFYFNGFSSGDKLEVLLGRTNEEKGFFKAKLDYVRKPIPETVIVKLLEKLYNEDVGLGLIQMYPYGGKMDEIPESAIPFPHRVGFIYKILYLSQWEKEEEGERHLNWVRSVYNYMTPFVSKSPRASYLNYRDFDLGTNNKNGPTSYGQASIWGKKYFDKNFKRLVHVKTKVDPTNFFRNEQSIPPLSVRGL

SEQ ID NO: 26 - protein, 603 aa, Artificial Sequence
  Description of Artificial Sequence Synthetic polypeptide
MRFPSIFTTVLFAASSALAAPVNTTTEDETAQIPAEAVIGYLDLEGDFDVAVLPFSNSTNNGLLFINTTIASIAAKEEGVSLDKRANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 27 - protein, 607 aa, Artificial Sequence
  Description of Artificial Sequence Synthetic polypeptide
MRFPSIFTTVLFAASSALAAPVNTTTEDETAQIPAEAVIGYLDLEGDFDVAVLPFSNSTNNGLLFINTTIASIAAKEEGVSLDKRANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHHHDEL

SEQ ID NO: 28 - protein, 542 aa, Artificial Sequence
  Description of Artificial Sequence Synthetic polypeptide
MKFSAGAVLSWSSLLLASSVFAQQANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 29 - protein, 549 aa, Artificial Sequence
  Description of Artificial Sequence Synthetic polypeptide
MGQSRGILGGVRQLILVILVGAYLSRLSAVDANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 30 - protein, 544 aa, Artificial Sequence
  Description of Artificial Sequence Synthetic polypeptide
MRFPSIFTTVLFAASSALAAPVNTTTEDETAQIPAEAVIGYLDLEGDFDVAVLPFSNSTNNGLLFINTTIASIAAKEEGVSLDKRPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 31 - protein, 460 aa, Artificial Sequence
  Description of Artificial Sequence Synthetic polypeptide
MPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 32 - protein, 545 aa, Artificial Sequence
  Description of Artificial Sequence Synthetic polypeptide
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 33 - protein, 543 aa, Artificial Sequence
  Description of Artificial Sequence Synthetic polypeptide
MATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQPRMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYAS

SEQ ID NO: 34 - protein, 4 aa, Artificial Sequence
  Description of Artificial Sequence Synthetic peptide
HDEL

SEQ ID NO: 35 - protein, 4 aa, Artificial Sequence
  Description of Artificial Sequence Synthetic peptide
KDEL

SEQ ID NO: 36 - protein, 5 aa, Cannabis sativa
YTPRK